(19) United States
(12) Patent Application Publication
(10) Pub. No.: US 2006/0195128 A1
(43) Pub. Date: Aug. 31, 2006

(54) Method and apparatus for loading penetrating members

(76) Inventors: Don Alden, Sunnyvale, CA (US); Timothy Donald Barrow-Williams, St. Albans (GB); Charlie Michael Dean, New York, NY (US); Martin-Lawrence Hughes, Milton Keynes (GB); Mark Robson Humphries, Herts (GB); Douglas Ivan Jennings, Royston (GB); Thomas Neudeck, Cambridge (GB); John Antony Stephenson, Cambridge (GB)

Correspondence Address: HELLER EHRMAN LLP, 275 MIDDLEFIELD ROAD, MENLO PARK, CA, 94025-3506, US

(21) Appl. No.: 10/541,123

(22) PCT Filed: Dec. 31, 2003
(86) PCT No.: PCT/US03/41747

§ 371 Date: Mar. 31, 2006

Related U.S. Application Data
(60) Provisional application No. 60/437,359, filed on Dec. 31, 2002.
(60) Provisional application No. 60/437,205, filed on Dec. 31, 2002.
(60) Provisional application No. 60/478,661, filed on Jun. 13, 2003.

(51) Int. Cl. A61B 17/32

(52) U.S. Cl. 606/181

(57) ABSTRACT

A body fluid sampling system is provided. In one embodiment, the system may comprise a penetrating member driver, a plurality of penetrating members sufficient for penetrating tissue; a tape coupling together at least two of the penetrating members; and a penetrating member release device removing the penetrating member from a sterile environment prior to use and moving the penetrating member into position to be operatively coupled to the penetrating member driver. The driver may comprises of a drive force generator for advancing the penetrating member and a processor coupled to the drive force generator capable of changing the direction and magnitude of force exerted on the penetrating member during the lancing cycle. The driver may further include a human interface on the housing providing at least one output for communicating with the patient.

### BACKGROUND OF THE INVENTION

Lancing devices are known in the medical health-care products industry for piercing the skin to produce blood for analysis. Typically, a drop of blood for this type of analysis is obtained by making a small incision in the fingertip, creating a small wound, which generates a small blood droplet on the surface of the skin.

Early methods of lancing included piercing or slicing the skin with a needle or razor. Current methods utilize lancing devices that contain a multitude of spring, cam and mass actuators to drive the lancet. These include cantilever springs, diaphragms, coil springs, as well as gravity plumbs used to drive the lancet. The device may be held against the skin and mechanically triggered to ballistically launch the lancet. Unfortunately, the pain associated with each lancing event using known technology discourages patients from testing. In addition to vibratory stimulation of the skin as the driver impacts the end of a launcher stop, known spring based devices have the possibility of firing lancets that harmonically oscillate against the patient tissue, causing multiple strikes due to recoil. This recoil and multiple strikes of the lancet is one major impediment to patient compliance with a structured glucose monitoring regime.

Another impediment to patient compliance is the lack of spontaneous blood flow generated by known lancing technology. In addition to the pain as discussed above, a patient may need more than one lancing event to obtain a blood sample since spontaneous blood generation is unreliable using known lancing technology. Thus the pain is multiplied by the number of attempts required by a patient to successfully generate spontaneous blood flow. Different skin thickness may yield different results in terms of pain perception, blood yield and success rate of obtaining blood between different users of the lancing device. Known devices poorly account for these skin thickness variations.

A still further impediment to improved compliance with glucose monitoring are the many steps and inconvenience associated with each lancing event. Many diabetic patients that are insulin dependent may need to self-test for blood glucose levels five to six times daily. Older patients and those with deteriorating motor skills encounter difficulty holding the many small parts such as lancets or test strips associated with using one of the known testing devices. The loading and unloading of lancets in known devices also exposes a patient to the handling of clean sharps and used sharps. Some patients also, knowingly or unknowingly, use the same lancet for multiple lancing events. At an extreme, some patients simply do not change the lancet at all.

Known lancing systems fail to adequately address these issues associated with ease of use, level of lancing pain, or other drawbacks that currently limit the compliance of diabetic patients with their blood glucose monitoring regime. Additionally, these known lancing systems fail to be suitable for a population of diabetic patients who may be of extended age and may be challenged in following proper lancing procedure.

### SUMMARY OF THE INVENTION

The present invention is directed at solving at least some of these issues. The present invention provides a multiple penetrating member solution for body fluid sampling. Specifically, the present invention provides systems, devices, and methods for removing penetrating members from penetrating member enclosures and loading the penetrating members onto a penetrating member driver. One embodiment of such a device may allow patient to perform multiple lancing events without having the patient manually insert a penetrating member into the penetrating member driver. At least some of these and other objectives described below will be met by embodiments of the present invention.

In one embodiment, an integrated device is provided that individually removes penetrating members from sealed storage and places the penetrating member into a transfer drum. In some embodiments, the sealed storage may comprise blisters or other enclosures. The penetrating member is then positioned in line with an actuator and is driven into the user's skin to create an injury that produces a small amount of blood for analysis. The used penetrating member is removed from the penetrating member coupler and placed in a safe storage container for eventual disposal.

Designing a suitable tissue penetrating system for diabetic patients requires overcoming several hurdles. Such a system would need to address issues of penetrating member esterility, ease of use, packaging, penetrating member actuation, penetrating member transport, and the like.

The present invention provides a continuous feed of multiple sterile penetrating members in a single connected packaging, where the packaging is advanced to sequentially present new sterile penetrating members for use with a driver. This advantageous minimizes user handling of the penetrating member during operation of the device.

In one embodiment, a body fluid sampling system is provided. The system may comprise a penetrating member driver; a plurality of penetrating members sufficient for penetrating tissue; a tape coupling together at least two of the penetrating members; and a penetrating member release device removing the penetrating member from a sterile environment prior to use and moving the penetrating member into position to be operatively coupled to said penetrating member driver.

A further understanding of the nature and advantages of the invention will become apparent by reference to the remaining portions of the specification and drawings.

### DESCRIPTION OF THE DRAWINGS

FIGS. 1 and 2 show internal components of one embodiment of a tissue penetrating device.

FIGS. 3 and 4 show one embodiment of a penetrating member loading and unloading assembly.

FIG. 5 shows another embodiment of a penetrating member loading and unloading assembly.

FIG. 6A show perspective and exploded views of one embodiment of a tissue penetrating device.

FIGS. 7A-7E show other embodiments of the present invention.

FIGS. 8A-8B and 9A-9B show various views of penetrating members and a loading and unloading assembly.

FIGS. 10A-10B show various embodiments of spools according to the present invention.

FIGS. 11A-11B show internal and external views of one embodiment of a loading and unloading assembly and a cartridge.

FIGS. 12-17 show various geometries used to obtain peeling of tape to expose penetrating members.

FIG. 18 is a cross-sectional view of one embodiment of a chuck according to the present invention.

FIGS. 19-20 show various embodiments of rotating drums according to the present invention.

FIGS. 21A-21B show one embodiment of a differential according to the present invention.

FIGS. 22-24 show internal components of various embodiments of a tissue penetrating device and a loading and unloading assembly.

FIGS. 25-26 show various orientations of penetrating members on a tape.

FIG. 27 illustrates an embodiment of a controllable force driver in the form of a cylindrical electric lancet driver using a coiled solenoid-type configuration.

FIG. 28A illustrates a displacement over time profile of a lancet driven by a harmonic spring/mass system.

FIG. 28B illustrates the velocity over time profile of a lancet driver by a harmonic spring/mass system.

FIG. 28C illustrates a displacement over time profile of an embodiment of a controllable force driver.

FIG. 28D illustrates a velocity over time profile of an embodiment of a controllable force driver.

FIG. 29 is a diagrammatic view illustrating a controlled feed-back loop.

FIGS. 30-34 show various views of one embodiment of a loading and unloading assembly in use.

FIGS. 35-37 show various views of another embodiment of a loading and unloading assembly in use.

FIGS. 38-41 show various views of another embodiment of a loading and unloading assembly in use.

FIGS. 42-44 show various views of another embodiment of a loading and unloading assembly in use.

FIG. 45 shows yet another embodiment of a loading and unloading assembly in use.

FIGS. 46-53 show still further embodiments of assemblies according to the present invention for loading penetrating members.

FIGS. 54A-54C show one embodiment of an enclosure for holding a penetrating member in a sterile condition.

FIGS. 55A-55B show another embodiment of an enclosure for holding a penetrating member in a sterile condition.

FIGS. 56 and 57 show various tractor holes according to the present invention.

FIGS. 58A-58C shows various orientations of penetrating members on a tape.

FIG. 59 shows yet another embodiment of a penetrating member loading and unloading assembly.

FIGS. 60A and 60B show embodiments of devices for storing used penetrating members.

### DESCRIPTION OF THE SPECIFIC EMBODIMENTS

The present invention provides a multiple penetrating member solution for body fluid sampling. Specifically, the present invention provides systems, devices, and methods for removing penetrating members from penetrating member enclosures and loading the penetrating members onto a penetrating member driver. One embodiment of such a device may allow patient to perform multiple lancing events without having the patient manually insert a penetrating member into the penetrating member driver.

It is to be understood that both the foregoing general description and the following detailed description are exemplary and explanatory only and are not restrictive of the invention, as claimed. It must be noted that, as used in the specification and the appended claims, the singular forms “a”, “an” and “the” include plural referents unless the context clearly dictates otherwise. Thus, for example, reference to “a material” may include mixtures of materials, reference to “a chamber” may include multiple chambers, and the like. References cited herein are hereby incorporated by reference in their entirety, except to the extent that they conflict with teachings explicitly set forth in this specification.

In this specification and in the claims which follow, reference will be made to a number of terms which shall be defined to have the following meanings:

“Optional” or “optionally” means that the subsequently described circumstance may or may not occur, so that the description includes instances where the circumstance occurs and instances where it does not. For example, if a device optionally contains a feature for analyzing a blood sample, this means that the analysis feature may or may not be present, and, thus, the description includes structures wherein a device possesses the analysis feature and structures wherein the analysis feature is not present.

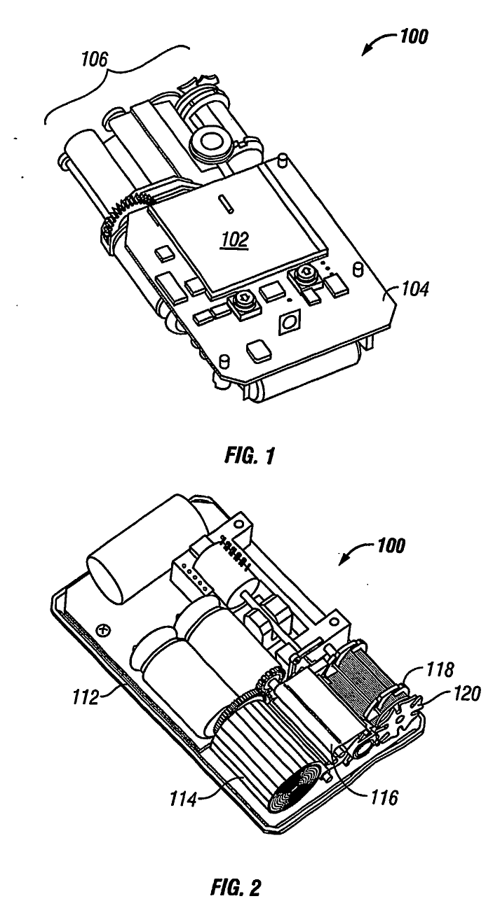

Referring now to FIGS. 1 and 2, one embodiment of a tissue penetrating system 100 is shown. Any of the penetrating member feed mechanisms or loading mechanisms of the present application may be incorporated and/or adapted for use with the tissue penetrating system 100. FIG. 1 shows internal workings of one embodiment of the tissue penetrating system 100 with an LCD display 102, pc board 104, and the penetrating member loading and unloading assembly 106. In some embodiments, the assembly 106 is enclosed in a cartridge or housing to facilitate handling. FIG. 2 shows the other side of the system 100. From this view, one can see an embodiment of the electromechanical penetrating member driver 110, capacitors 112, a spool 114 of tape 122 having unused penetrating members, a tape peeling roller 116, a penetrating member drive chuck 118, and a used penetrating member drum 120.

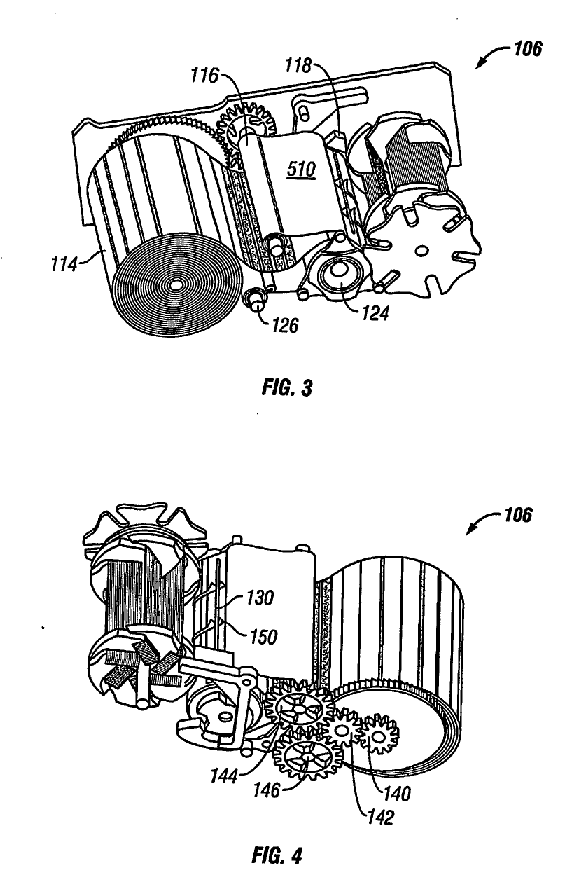

FIGS. 3 and 4 show further details on one embodiment of the penetrating member loading and unloading assembly 106. The tape 122 may be wound into a spool 114 with the tape being fed towards a first roller 124. Tape peeling rollers 116 and 126 collect the used tape after the tape is peeled and penetrating member removed. In one embodiment, the tape 122 may comprise of a bottom layer and a top layer with the unused penetrating members held therebetween. The tape 122 may fully enclose each of the penetrating member. In other embodiments, the tape 122 only partially covers each penetrating member and may cover those portions that penetrate the patient and are to remain sterile prior to use. Although not limited to the following, the tape used with the present invention may be a flexible support band/ribbon/strip, etc. which has a number of functions, including: may comprise of a number of different materials/layers; have dimensional stability (longitudinally and laterally); maintaining a sterile environment/low permeability about at least a portion of the penetrating member, features in known locations (penetrating member relative to each other; penetrating members relative to the device/drive mechanism; features to drive the tape); formable, such as but not limited to preformed pockets on the tape; compliant/conforming, to form around the penetrating members if we don't perform; sufficiently flexible to allow it to be coiled, without degrading its integrity; thin to reduce storage volume; Low friction; as a sandwich structure, upper and lower ‘films’ can be of a different material/combination/construction; exhibiting pierce resistance and/or tear resistance; ability to post-sterilize without degradation of properties; or convenient structure to manufacture the base material, manufacture the loaded/sealed structure, assemble into the device and then use in the device. In one embodiment, the design of the tape comprises two strips of material, one of which (base web) is formed into a series of depressions to hold the penetrating members, with the second, flat strip (top web) sealed to the top surface to trap the penetrating members into the individual depressions or pockets.

In one embodiment, the materials used for the tape have to perform several functions, including but not limited to: 1) maintaining the penetrating member in a sterile condition from packaging/sterilisation to the point of opening; 2) resisting puncturing by the penetrating member point during packing, transportation, and storage within the device; 3) holding the pockets (and penetrating members) in registered position relative to the opening mechanism, so that transfer is reliably achieved; 4) peeling open with a controlled, predictable force consistent with the mechanism of the device; 5) retaining predictable properties (especially peeling force) after sterilisation of the penetrating members in the bandolier; 6) using the minimum storage volume for the filled tape and the two strips of packaging after the penetrating members have been removed; and/or 7) avoiding contamination of the penetrating members by fugitive ingredients of the tape material. This implies the use of medical/pharmaceutical grade materials.

In one embodiment, the tape 122 may be made from a variety of materials including but not limited to a polymer or multiple layered materials. A range of materials include but are not limited to: 1) polyamide/polyethylene, thermoformable base webs (for example MW100 with a thickness of 100 mm supplied by Wipak; or a 200 mm nylon/LDPE film from Rexam Medical) (in both these films, the nylon component provides puncture resistance, while the polyethylene allows the film to be heat sealed to a range of top webs); 2) an 80 mm polypropylene, thermoformable base web (DUS4 from Westfield Medical) offering low material cost with lower thickness than the complex films containing nylon; 3) a 150 mm EVA/Surlyn/EVA film from Perfecseal, for use as both thermoformable base and plain top webs. The Surlyn is an alternative puncture-resistant layer to nylon; 4) a 62 mm polyester (PET)/polyethylene top web (for example ESE 1250 supplied by Wipak) (the polyester outer layer provides dimensional stability, and allows high quality printing on the top face of the tape if required); 5) a range of gas-permeable papers for use as the top web (these provide a good print surface with the option of either ethylene oxide or irradiation sterilisation methods. Material examples may include 60 gsm Gas Paper 60 supplied by Wipak; Tyvek 10739 with a grid pattern sealing lacquer supplied by Rexam Medical; 60 mm Superpeel and 63 mm Superseal medical papers, both supplied by Westfield Medical); 6) aluminum foils with a plain lacquer on the outside and a thin layer of heatseal lacquer on the inside (the foils are either hard or soft tempered, and allow cold forming of the pockets in the base web with a good print surface on the top web and examples include 30 mm and 38 mm Omniseal from Chadwicks of Bury); and 7) aluminum foil laminates comprising a metal foil combined with one or more layers of plastic (these products may be cold formable, but the plastic helps to minimize puncturing of the metal layer by the penetrating member, provides a tough composite with good peeling characteristics, and may also act as the heat sealing layer).

Many combinations of metal foil thickness, metal temper, plastic films and lacquers are possible. As an example the following combination supplied by Hueck Folien is of interest: 1) base web: lacquer/30 mm soft temper aluminum/adhesive/30 mm coextruded PP; and 2) top web: lacquer/30 mm soft temper aluminum/adhesive/25 mm PP film. These materials may be used for packing scalpel blades, and can be peelably-sealed to each other.

Other potentially useful laminates include: 1) lacquer/7 mm soft temper aluminum/adhesive/25 mm PP film; and 2) 23 mm PET/adhesive/20 mm soft temper aluminum/adhesive/50 mm coextruded PP. Again, these examples and those listed above are purely exemplary in nature and are nonlimiting. Multiple layer embodiments may confer different properties, eg. mechanical strength/robustness, barrier properties, etc, and specific material choices/thicknesses to give these properties. The tape 122 may have a coating such as but not limited to silicone. Other low function coatings on the outside of the tape, including low friction materials and/or coatings, may be used. Different materials may be used on the inner and outer surfaces of tape 122. Different materials may be used on the upper and lower layers of the tape 122. The tape 122 may have elasticity and cutting properties such as but not limited to resistance to tearing or resistance to piercing, depending on the desired quality. The tape 122 may also be sufficient to provide a sterile environment about the covered portions of the penetrating member over certain environmental conditions (temperature, humidity, etc . . . ) for a period of time such as but not limited to at least about 24, 12, 8, 6, 3, 2, or 1 months. Different areas on the tape may be sterile and non-sterile areas. Surfaces over which the tape moves may also be covered with materials of different properties.

The penetrating members may be housed in pockets on the tape. The pockets may be similar to blister packs or they may be vacuum sealed or substantially flattened against the penetrating member. The pockets may also be compressed or squashed to more space efficiently wind the tape 122 about a storage spool. Minimizing the radius of coil may involve one of the following: radius may be minimized through the mechanism of attachment to the wind up spool, diameter of wind up spool, bend radius of the material, resistance/tension of film, film thickness, elasticity of film (inner/outer diameters), no backlash in the drive/spool. The seal zone about each pocket may be controlled for peels strength that may be uniform or deliberately variable. The pockets on the tape 122 may be varied in shape, such as but not limited to being narrowed at locations where a blade 150 will engage the tape, such as but not limited to an hourglass configuration. If desired, the pocket may also be widened at areas with the blade 150 will engage the tape. The tape 122 may have varying width. Instead of a tape with constant width, the width may be deliberately varied or it may be tapered. The tape 122 may be printed with information such as but not limited to a penetrating member counter or display other properties about the penetrating member. The tape 122 may include sensors, markings, contours, openings, coloring, or other indicator for relaying counting, penetrating member presence, penetrating member location, or other information.

FIG. 3 shows a penetrating member 130 held in a launch position, ready to be actuated by the drive chuck 118. After actuation, the penetrating member is moved to a used penetrating member drum or container 120. In this particular embodiment, the used penetrating member drum 120 is indexed using a Geneva wheel mechanism which positions the slots 121 on the drum in position the receive each used penetrating member. Coil springs on the drum 120 are used to urge the used penetrating member or penetrating member towards the center of the drum. This provides for more efficient storage of the penetrating members. It should be understood of course that coil springs could be expanded to any compliant, flexture which will urge the penetrating members into the preferred location in the drum and keep them there.

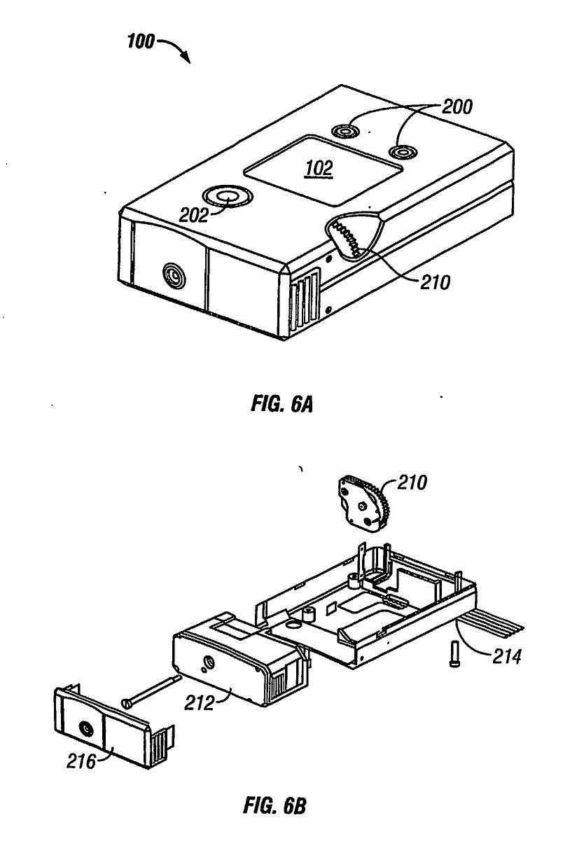

FIG. 4 shows a back side of another embodiment of assembly 106. The gears 140, 142, 144, and 146 operate to move penetrating members from the spool 114, peel away the protective tape 122, and position the penetrating member to be actuated by the drive chuck 118. In a further embodiment, a differential 132 (FIG. 6) is used to balance the torque on the takeup spools. This differential operates in a manner similar to a torque wrench where a user driven or powered wheel is used to drive the gears 140, 142, 144, and 146. The torque is maintained so that tension of the tape is controlled. The tension control and the differential will be described in FIGS. 6A and 6B. In some embodiments, the differential allows the relative tension between the engaged drive for the take up spools for both tapes to be equilibrated, making allowance for any non uniformity in the tape paths, lengths or encountered friction. Details of one embodiment of the differential are shown in FIG. 21. It should be understood that in some embodiments, the gears can be fixed with respect to each other, independently driven or used with the differential.

Of course, other numbers of gears and other configurations/combinations of devices may also be used. First roller 124 has penetrating member holders 150 which may pierce the tape and define a slot, groove, two holes in alignment, or contour for slidably receiving each penetrating member. The slot acts as a penetrating member guide or bearing during actuation of the member by the drive chuck 118 to penetrate tissue. After actuation, the penetrating member is returned into the apparatus and may be moved to some storage area such as but not limited to the used penetrating member drum 120 or another storage device. As seen in FIG. 4, the penetrating members themselves may have a variety of configurations such as but not limited to an L-shape configuration, with a bent proximal end to facilitate handling of the bare penetrating member.

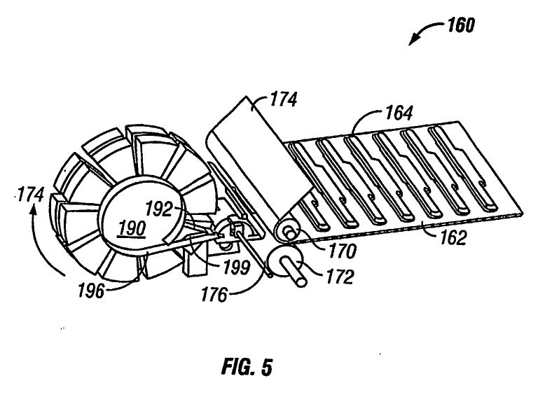

Referring now to FIG. 5, another embodiment of the present invention shows a penetrating member loading and unloading assembly 160 similar to the assembly of FIGS. 3-4. The assembly 160 has a tape 162 having a plurality of enclosures 164 containing penetrating members for use with the tissue penetrating system. A first roller 170 and a second roller 172 are positioned to peel a top layer 174 away from a bottom layer 176 of the tape 162 to release a penetrating member 178 enclosed therein. The penetrating member 178 is then positioned for use with the drive chuck 180. After use, the used penetrating member is moved towards the storage device 190. As the penetrating member 178 is picked up by slot 192, the rotation of the storage device 190 as indicated by arrow 194 will place the penetrating member into contact with guide 196. The storage device 190 rotates to urge the penetrating member in the direction indicated by arrow 598 against the guide 196 while the penetrating member remains in slot 192. The rotational motion of the storage device 190 and use of a guide 196 urges all of the used penetrating members in the slots towards the center of the storage device, eventually arranging them in the manner shown in FIG. 4.

In the embodiment shown in FIG. 5, the guide 196 has a coiled, curved portion near the center of the device 190 and an elongate portion. In some embodiments the elongate portion extends out to the outer diameter of the storage device, extends beyond the outer, or may be at some other distance sufficient to engage penetrating members and guide them towards the center of the storage device 190. As the number of penetrating members held in the storage device 190 increases, the diameter of the coiled portion of the guide 196 will also increase. The elongate portion will continue to extend radially outward to engage freshly used penetrating members and urge them towards the center of their respective slot. The guide 196 may be placed on either the distal end or the proximal end of the storage device 190, guides may be placed on both ends, or a plurality of guides may be used.

Referring now to FIGS. 6A and 6B, an assembled and partially disassembled embodiment of the tissue penetrating system 100 is shown. FIG. 6A shows the system 100 with a interface 102 displaying information about the system 100, buttons 200 for changing settings or other functions, and a launch button 202 for actuation of the penetrating member. A user turnable wheel 210 is provided for indexing the penetrating member, removing a used member and loading a new member into launch position.

FIG. 6B provides a partially disassembled view of the system 100. For ease of illustration, the driver, interface 102, upper component, and other parts are not shown. FIG. 6B does show the wheel 210, cartridge 212, a lower cover 214, and a front cover 216. The cartridge 212 contains the loading and unloading assembly 106 that moves unused penetrating members into position for actuation and then into a storage area. In this embodiment, the wheel 210 is coupled to the cartridge 212 for indexing purposes.

The wheel 210 may be used to control tension in the tape of unused penetrating members housed in cartridge 212. A differential (not shown) coupled to the wheel 210 or inside the cartridge 212 may be used to balance the torque on the takeup spools of the tape in the cartridge. This differential operates in a manner similar to a torque wrench. A user can turn the wheel 210 to remove an slack in the tape and maintain a desired level of tension in the tape. In one aspect, this ensures that when the penetrating member or penetrating member is removed from the tape, the tape is pulled against the drum and not still adhered to the penetrating member. In another aspect, maintaining tension in the tape prevents slack in the tape from causing the tape to jam the device. Tension control also ensures that the device remains synchronized so that the penetrating members are positioned to arrive at a location where the penetrating member loading device may engage it. The differential or torque wrench like device allows the user to apply optimal amount of pressure to maintain tension control of the tape. If the user applies too much force or turns the wheel 210 too many times, the wheel 210 will slip and not wind the tape beyond a predetermined level of tension. FIG. 6B shows the differential as 210, ie. It is more than the indexing wheel. It controls the tape tension of the unused part of the system through the take up spools for the upper and lower tapes. Another construction may be developed where the drive wheel drove the blade drum, and the take up spools were held in over tension.

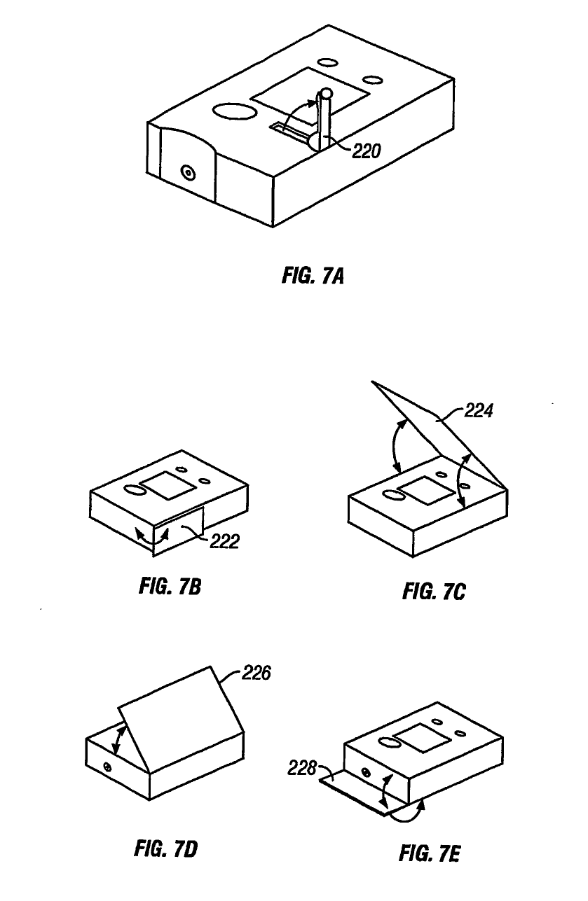

Referring now to FIGS. 7A-7E, a variety of devices may be used to facilitate the indexing of a used penetrating member and the loading of a new one. FIGS. 6A and 6B show the use of a wheel 210 such as but not limited to a thumb wheel, finger wheel, or the like. The size, width, diameter, texture, protrusion, teething, and the like may be altered on wheel 210 to facilitate indexing. FIG. 7A shows that a lever 220 may be used to increase the mechanical advantage. FIG. 7B shows that a cover 222 removable from the launch portion of the system 100 can also be used for indexing. The movement of the cover 222 causes rotation that may be used to turn a differential inside the system 100. The opening of top covers 224 and 226 as seen in FIGS. 7C and 7D may also be used to provide increased mechanical advantage for indexing or for ease of use. FIG. 7E shows a still further embodiment where a front cover 228 is hinged and is movable to provide rotational force that may be used to turn gears and parts inside the system 100. These covers 222, 224, 226, and 228 may be configured so that they lie flat against a surface of the system 100.

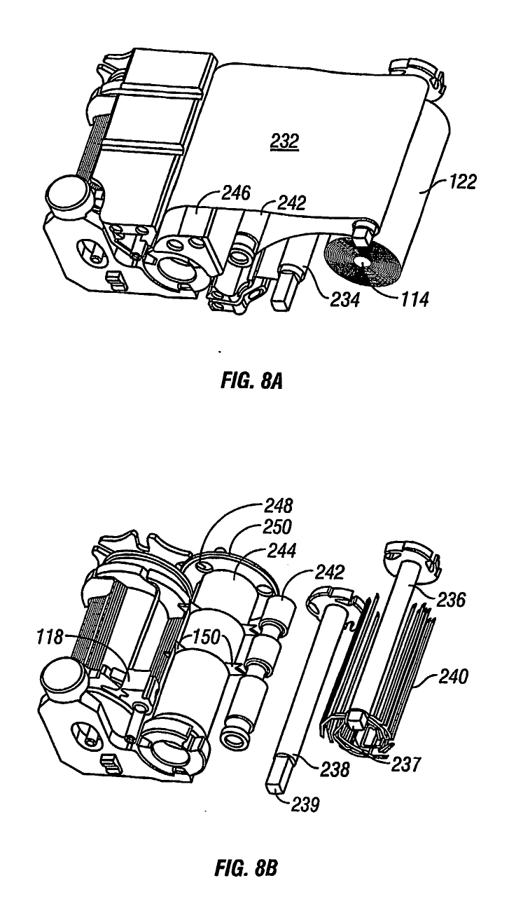

FIGS. 8A and 8B show a still further embodiment of an assembly 230 according to the present invention. FIG. 8A shows a roll of tape 122 that is peeled apart into a first portion 232 and a second portion 234. The first portion 232 is wrapped around a first takeup spool 236 and the second portion 234 is wrapped around a second takeup spool 238. These spools are more clearly shown in FIG. 8B. The spools 236 and 238 may have a keyed portion 237 and 239 to allow those spools to be driven or to have gears or other devices attached to them. We can include both options—in one embodiment both spools can be driven. Spool 114 contains the supply of tape 122 having the unused penetrating members 240. FIG. 8B also shows a roller 242 and a penetrating member loading device 244 which in this embodiment is a transfer drum having blades 150 for engaging the members 240.

Referring again to FIG. 8A, tape 122 from the spool 114 is pulled toward roller 242 and pulled apart with the first portion 232 pulled against a peel surface 246 and the second portion 234 against the loading device 244. Roller 242 was included to optimise the geometry of the tape as it was fed round the drum blade. It increased the amount of tape wrapped round the drum allowing more than one set of blades to engage the tape at any one time, and align with the next penetrating member. In this embodiment, the peel surface 246 does not rotate. In other embodiments, the peel surface 246 may rotate and may be a roller. The surface was used as it could be integrated into the device, was a fixed geometry and helped define the position of the peel point. At one stage we considered using a roller system to try to achieve a similar function, as we considered it might reduce friction as it was a moving element. As will be discussed below, the penetrating member 240 of the tape peeling apparatus will be engaged by one of the blades 150 on the loading device 244 prior to the penetrating member being fully released from both tape portions 232 and 234. Thus in this embodiment, the member 240 is not disengaged from the tape and free to move until it is coupled to the blade 150. After the penetrating member 240 is engaged by blade 150, the penetrating member is rotated to engage the drive chuck 118 which actuates the member to move out of the opening 248 as indicated by arrow 250.

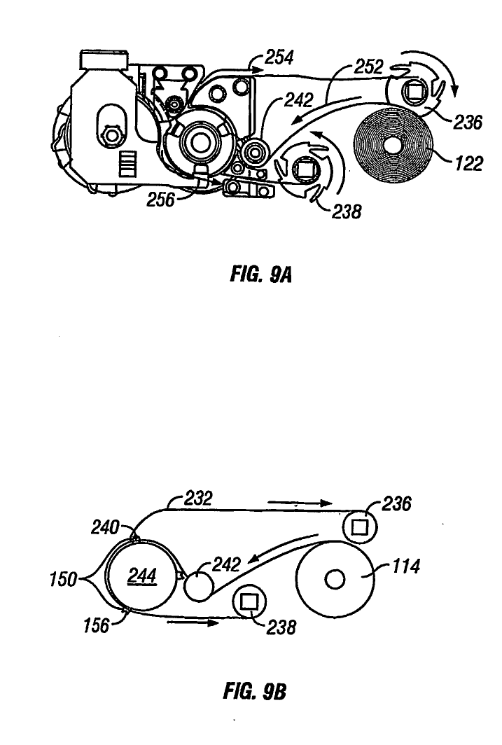

FIG. 9A more clearly shows the path of the tape 122 as it travels through this embodiment of the tape peeling apparatus. As indicated by arrow 252, the tape 122 is first drawn towards roller 242. As the portions are peeled apart, arrow 254 shows the portion 232 being drawn to takeup spool 236 while arrow 256 shows the portion 234 being drawn toward takeup spool 238.

FIG. 9B more clearly shows that the blade 150 in this embodiment of the invention has engaged the penetrating member 240 by the time the first portion 232 is being peeled away from the second portion 234. This ensures that the penetrating member 240 will be engaged by the loading device 244 before the tapes are peeled back, fully releasing the penetrating member.

Maintaining the synchronization of the tape 122 carrying the penetrating members and the loading device 244 having blades 155 is desired. This allows the penetrating members 240 to be presented to the V-shaped or other shaped slot 156 in the blade 155 and seat the penetrating member in the blade. In the present embodiment, penetrating members 240 on the tape 122 are separated by a known distance on the tape. Synchronization is about maintaining that fixed spacing of the members as the tape 122 is to be engaged with the loading device 244. As described, the overview of events involves a penetrating member picked up by the piercing blades 150, piercing blades 150 run round a transfer drum 244 and presents the penetrating to the chuck 118, penetrating member gets actuated, penetrating member gets further swept around the drum 244, and penetrating member is placed in the storage bin. There is another set of blades 150 that picks up the next penetrating member and the process is repeated until all penetrating members are consumed or the cartridge 212 is removed from the system 100.

In one particular embodiment, the piercing of a pocket around the penetrating member 240 may occur as follows. It should be understood of course, that not all penetrating members 240 are designed to have a pocket about it. In this particular embodiment, the sterile pocket is maintained until the point of use. Initially, sterility comes form the pre-formed pocket and sealed tape 122. As the tape 122 is advanced, blade 150 engages the tape and pierces either side of the pocket, but does not open the pocket. As the tape 122 is advanced to the peel point, the tapes 122 is separated into two portions. The blade 150 also penetrates further into the pocket to engaged the penetrating member 240. At this point, the next unused penetrating member (penultimate member) has a blade 150 pierces on either side of the pocket, but sterility is maintained. This positions the blade 150 to engage penetrating member and the cycle is repeated.

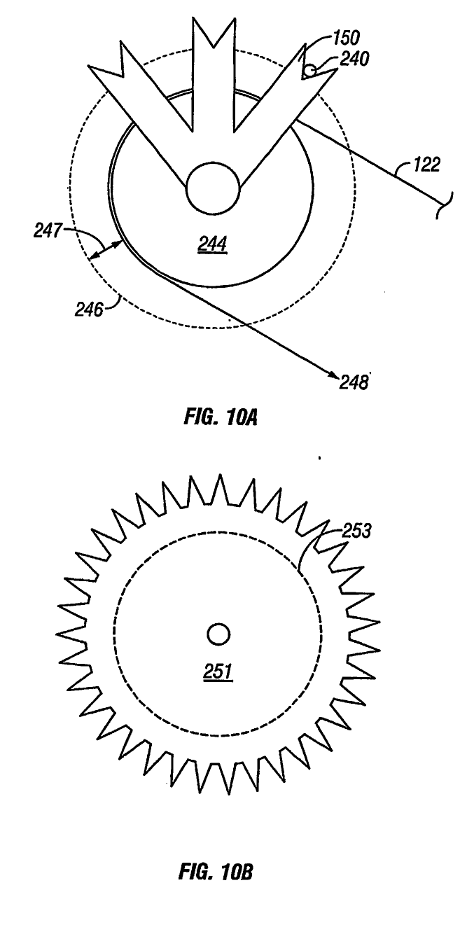

Referring now to FIG. 10, if there is slack in the tape 122, then the synchronization of the penetrating members and the loading device or transfer drum 244 may be disrupted. Slack in the tape 122 may also cause the assembly 106 to jam. The slack in the tape 122 may be caused by a slight path difference between the diameter that the penetrating member is going around the transfer drum 244 and the diameter that the tape is going around. As seen in FIG. 10, the path of the penetrating member 240 about the transfer drum 244 is indicated by dotted circle 246. The tape 122 is curved about the diameter created by drum 244, not that by the blades 150. In other words, the penetrating member 240 is now on a circumference running outside that of the tape. The tape 122 is running on the inside compared to where the bottom of the V groove on the inside of the blade 150. It is desirable, in this embodiment, that when the tape is peeled apart, the tape will also be separated at some point from the penetrating member 240. This prevents the tape from dragging on the penetrating member because it is still on the tape or next to the tape.

The dimensional difference 247, unfortunately, can cause slack to build unless the tape is tensioned as indicated by arrow 248. Relative slip between the sequence of blades 150 and location of penetrating members 240 in the tape 122 is undesirable because eventually, the tape will walk off and blade 150 will miss grabbing the next penetrating member that's coming into the drum 244. Slack can introduce such relative slip. The dimensional difference is compensated by controlling the tension so that penetrating members consistently arrive at the same location as the V groove on the drum 244. Besides pulling the tape tighter against drum, one could also cut more of the tape, or ride it around the blunt side of the blade to pull the tape forward. Various ideas were proposed to help address this issue. One was to make the V groove of the blade wide enough to accommodate some walk off of the system. We also considered having sharp edges along the length of the outside of the blade so any dimensional changes could be accommodated by a combination of tension on 248 and ‘slitting’ the tape by the leading/trailing edge of the blade, ie. You pulled the tape through under tension and let the tape slit to cope with any dimensional change. The other consideration was how to drive the tape. This could be done using tractor holes, but there were ideas round using a blunt edge to the blades and driving the tape using the blades. Both were implemented. Tractor holes or alignment holes in the tape may also be used. By running on tractor holes, synchronization will be maintained as long as the holes are engaged.

In the present embodiment, tension control is used to maintain synchronization. Tension control, control of the peel point through the geometry in the design, wide V grooves in the blades, dimensional matching of all the components, engagement of multiple blade sets, fixed drive in one embodiment, and differential in another, driving off the penetrating member tractor holes/pins. A differential 132 to get the tension to a predefined level and slip if the user winds it up beyond the predefined level. Thus as the used penetrating member 240 is being unloaded, the tape is also being tensioned so that the new penetrating member 240 will arrive to mate with a blade 150. The implication of poor tension control is that the peel point can move forward or backward, peel too late and get snagged in the mechanism, or peeled too early the penetrating member might be released from being held before being engaged by the chuck 118. As shown in the figures, the peel point where both tapes separate, but not necessarily where the penetrating member leaves the tape. In one embodiment, it is desirable that the tapes separate just as the leg of the a bent penetrating member is going into the chuck, so that the penetrating member is controlled. Good peel point control may allow a shorter bent portion on the penetrating member 240.

FIG. 10B show one embodiment of a transfer drum 251 designed to minimize the possibility of having a penetrating member 240 miss a V-groove on the drum. The inner circumference of the drum 251 is indicated by dotted lines 253 about which the tape is curved.

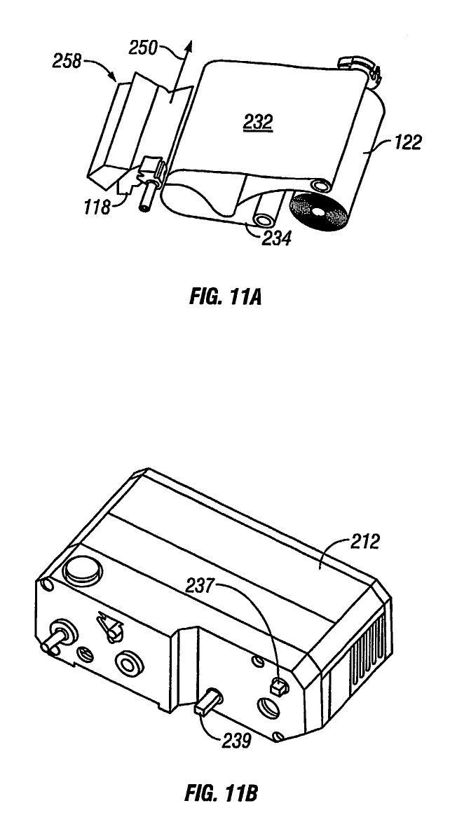

Referring now to FIGS. 11A and 11B, this embodiment of the present invention will be described further. FIG. 11A is an isolation view showing only the tape 122 and its portions 232 and 234 as they are separated. The rollers and peel surface have all been removed from illustration for ease of reference and to show how the tape travels through the tape peeling apparatus of the assembly. As the tape 122 is peeled apart, the penetrating member 240 (not shown) is soon presented to the drive chuck 118. In one embodiment, the chuck 118 has a slot for slidably receiving a portion of the penetrating member. The penetrating member 240 is actuated in the direction indicated by arrow 250 and then return back into the cartridge 212. The used penetrating members 256 may then be stored in configuration 258.

FIG. 11B shows one embodiment of the cartridge 212 housing the tape 122 and various parts of the assembly 106. As seen in FIG. 11B, the keyed portions 237 and 239 may extend outward from the cartridge 212, allowing drive elements such as but not limited to wheel 210 to turn the rollers. In some embodiments, gears or other connective drive device such as but not limited to belts or discs, may be used to allow these elements to be driven together, in a synchronized manner. There may also be an opening 260 allowing for the drive chuck 118 to be inserted into the cartridge 212 to engage penetrating members therein.

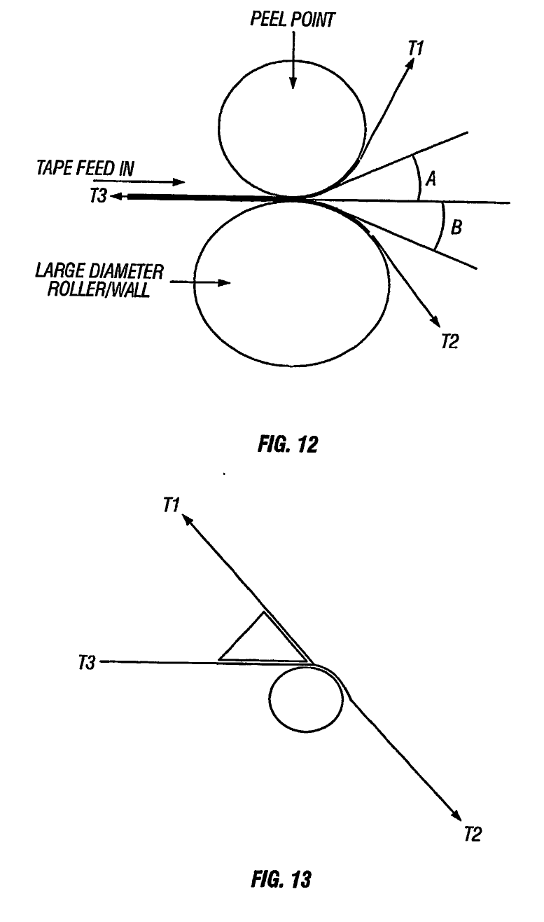

Referring now to FIGS. 12 and 13, various configurations for peeling the tape 122 apart will now be described. In some embodiments, it is also desirable to control the peel point, in addition to synchronizing arrival of penetrating member and blade. Controlling peel point may involve tension control, defining the geometry of how the two tapes are peel apart, consistent bonding on the tapes, pull apart in a controlled rate. In one embodiment, a range of pull apart angles may also be preferred. Different tension is also used for different geometry: there is a balance between the appropriate control tension and ease of use. Control tension a the point where you are indexing and removing the penetrating member.

FIG. 12 shows a peel point about rounded surfaces. Round surfaces were originally considered, as they could be moving parts reducing friction as the tape would not need to be dragged across the surface. Potentially they could also have features to drive the tape and be used to maintain synchronisation. To maintain a stable peel point tensions on the incoming tape and the two separated tapes would need to be maintained (ie. T1, T2 and T3) in FIG. 12.

FIG. 13 shows a peel point about a more pointed surface. An alternative configuration was to use a fixed part with a more ‘pointed surface’. In this instance the sharp edge geometry allows the peel point to be controlled within a small area, and with say 180° peel apart geometry, allows the peel point to be controlled through T1 and T2. This potentially removes the need to control T3 with a back tension, reducing the number of variables to be controlled and the total drag in the system.

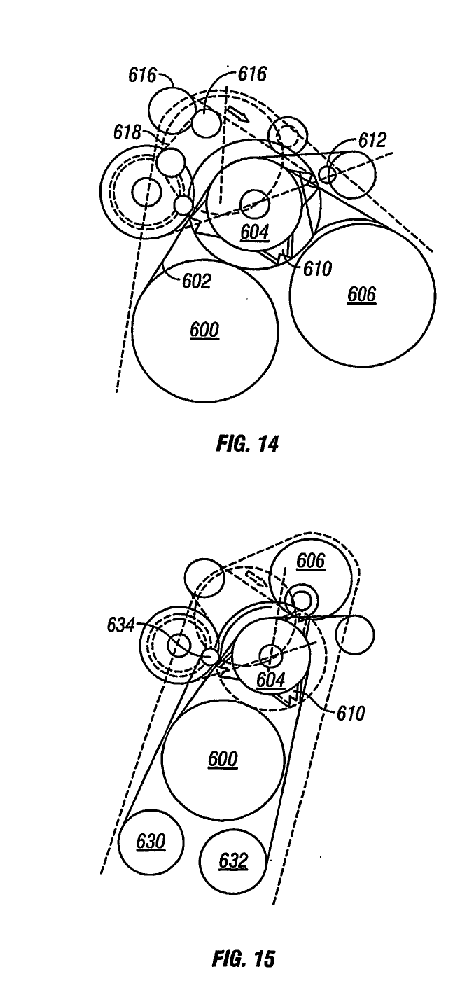

Referring now to FIGS. 14 and 15, various configurations are shown for the tape peeling apparatus for use with an assembly described herein. As seen in FIG. 14, an unused penetrating member store 600 holding tape 602 which wraps around roller 604 and is received by used penetrating member and tape store 606. In this embodiment, the roller 604 has tape piercing penetrating member guides 610 that penetrate the tape 602 to engage a penetrating member therein. Although not limited in this manner, in some embodiments this engagement by the piercing guide 610 occurs prior to the tape 602 being peeled or before the tape is fully peeled. In the embodiment of FIG. 6, a tape recombination pin or roller 612 is included. Thus in this embodiment, the used penetrating members are recombined with the tape and rolled into the used penetrating member and tape store 606. Rollers 614 and 616 may be used to assist the handling of one layer 618 of the tape while the penetrating member is positioned for actuation. After actuation, this layer 618 of tape is then recombined with the used penetrating members.

FIG. 15 shows another configuration of the tape peeling apparatus. The apparatus has a separate tape storage rollers 630 and 632 for containing used tape. A roller 634 is used to peel the tape back and to provide a pivot point for directing the tape to storage roller 630. In this embodiment, used penetrating members are directed to the storage area 606 and is not recombined with the tape.

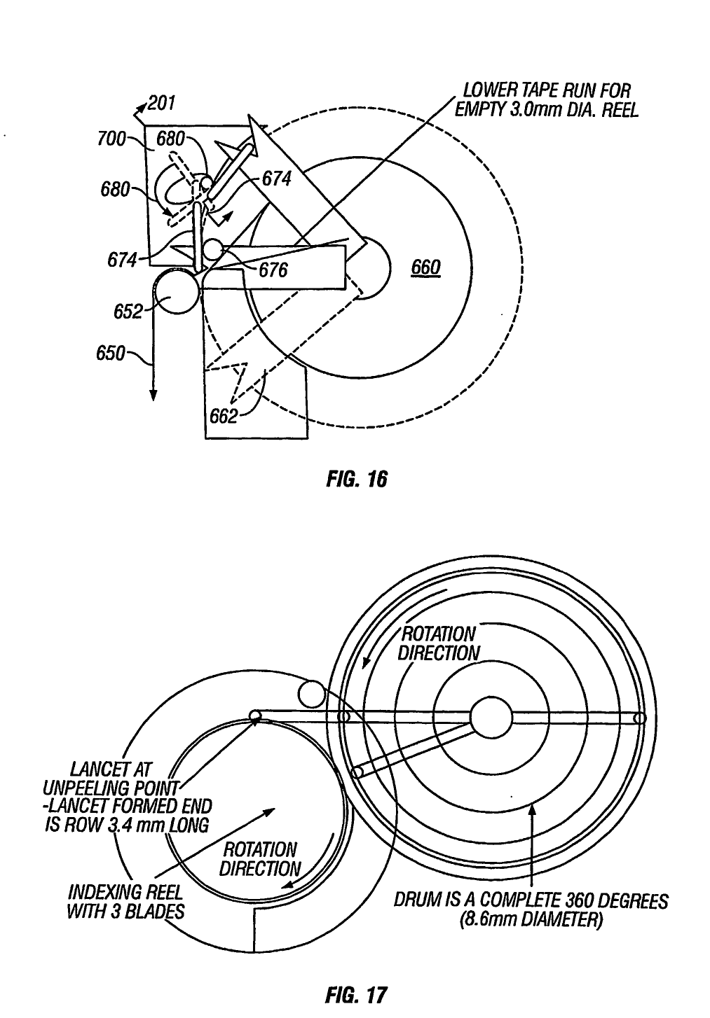

Referring now to FIG. 16, a still further embodiment of the tape peeling apparatus is described. A top layer 650 of the tape is wrapped around the roller 652 as the top layer is peeled off. The rotating reel 660 may have at least one piercing or penetrating member guide 662 which may engage a penetrating member in the tape prior to the penetrating member being fully released or removed from the tape. As seen in FIG. 8, the bottom layer 670 may be wrapped around the reel 660 which in this embodiment also serves to rotate the penetrating member guides 662. Tape position 671 show the initial position of the tape when not many penetrating members have been used and the spool of tape around the reel 660 has not been wrapped. FIG. 16 shows an end view of an L-shaped penetrating member 674 as it is released from the tape layers 650 and 670 while engaged by a penetrating member guide 662. A support member 676 may be provided to guide the bent portion of the penetrating member 674 as the penetrating member advances in the groove 678. As the penetrating member is transported by the guide 662, the penetrating member 678 may encounter a pivot bar or structure 680. As indicated by arrow 680, the bent portion of the penetrating member 674 is rotated to change the penetrating member orientation as the guide 662 is rotated. This may be used facilitate storage of the penetrating member as it is delivered to a used penetrating member store (not shown). A trailing leg orientation of the penetrating member or penetrating member transports more easily and tends not to snag or get caught as it is moved. The reason for the flip is to engage the drive chuck as described in FIG. 10 and then pull free for storage.

FIG. 16 also illustrates the peel point control which is related in part to maintaining tension on the tape. The peel point should be maintained in a consistent location so that the penetrating member is not released from the tape too early or too late. The peel point may be controlled by using tape having a consistent force requirement for peeling. It may also be controlled by having the rollers apply constant levels of force to the tape.

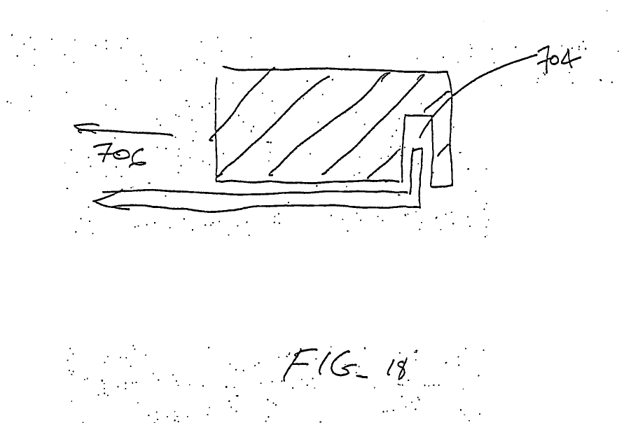

FIG. 17 shows a still further configuration for a penetrating member loading and unloading assembly. This is a version of a drive chuck that uses a continuous rotation drum. The drums in this embodiment rotate in the same direction. The drums may also be configured to rotated in opposite directions as defined by arrows on the drums. The drum may also be sided to have a reciprocating penetrating member pick off, instead of a continuous one. In continuous pick off, the drum rotates only in one direction. In a reciprocating design, the drum will rotate in one direction load the penetrating member and then rotate in an opposite direction to unload the penetrating member.

FIG. 18 shows a cross-sectional view of one embodiment of the drive chuck 700. As the penetrating member 702 is rotated, a bent portion of the penetrating member 702 is rotated upward into a slot 704. This allow the chuck 700 to drive the member 702 in the direction indicated by arrow 706.

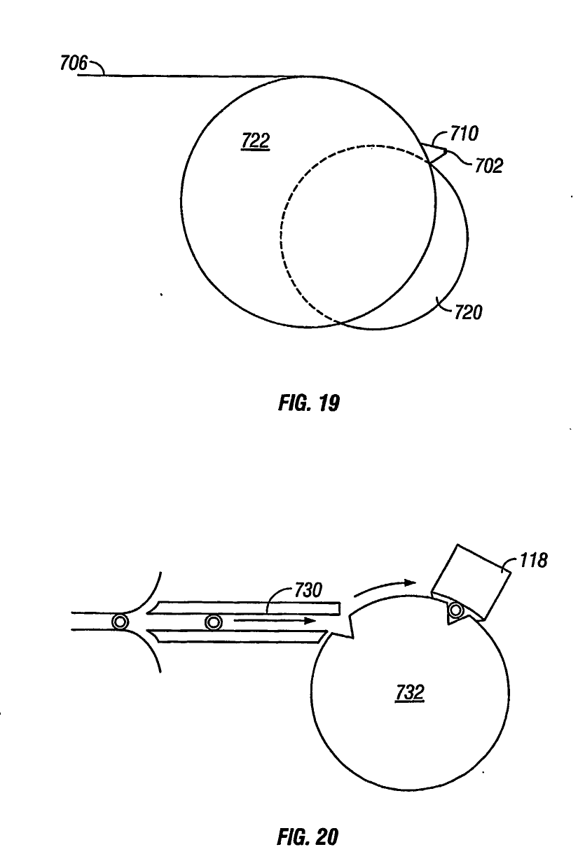

FIG. 19 shows a further embodiment where two rollers with at least one having an off centered drum will ensure that the tape 706 is pulled away from the penetrating member 702 held in a cutting blade 710. The drum 720 has an off-centered axis of rotation. In this embodiment, drum 722 has a centered rotation area. It should be understood, however, that if desired, this orientation may be changed so that drum 722 has the off centered axis of rotation and that the drum 720 has a centered rotation. In some embodiments, both drums may have centered or off-centered axis of rotation. In this embodiment, the intention here is to try to maintain a constant radius for the tape and the penetrating member to maintain synchronization; they can follow separate paths with a common distance traveled.

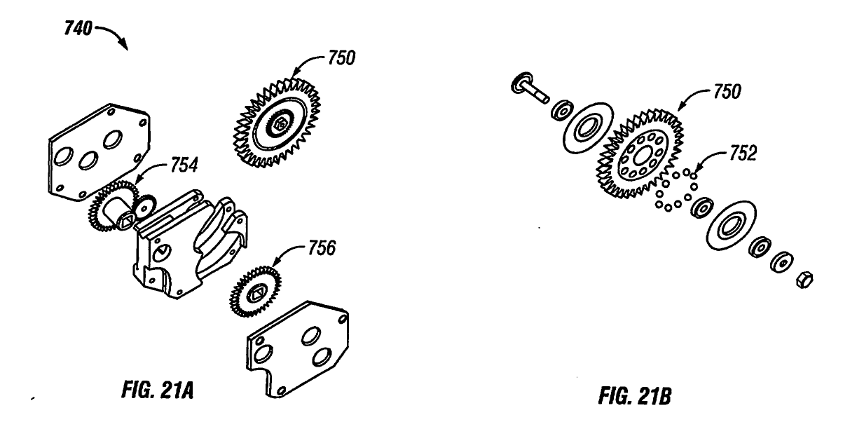

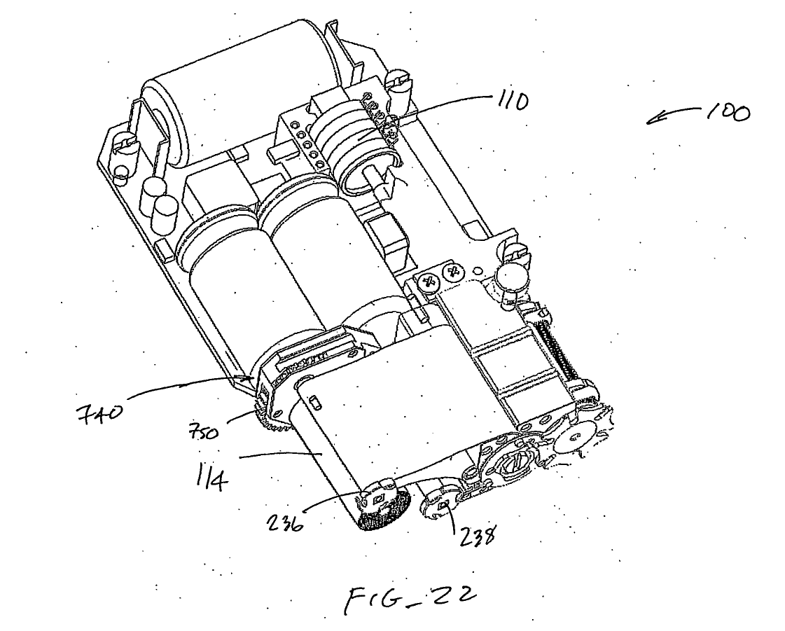

FIG. 20 shows a still further embodiment where a channel 730 is used to guide a penetrating member 240 to a rotating drum 732 which rotates to bring the penetrating member to the drive chuck 118. The channel 730 may be a flat surface or it may include a conveyor belt or other transport device to urge the penetrating member towards the drum 732.

FIG. 21A shows one embodiment of a differential 740 for use with the present invention. A main drive wheel 750 is provided. As seen in FIG. 21B, the wheel 750 includes a plurality of ball bearings 752 which allow the wheel 750 to slip and to compensate for excess tension in the system. As seen in FIG. 21A, the wheel 750 may drive two tooth gears 754 and 756 which may in turn drive take up spools 236 and 238.

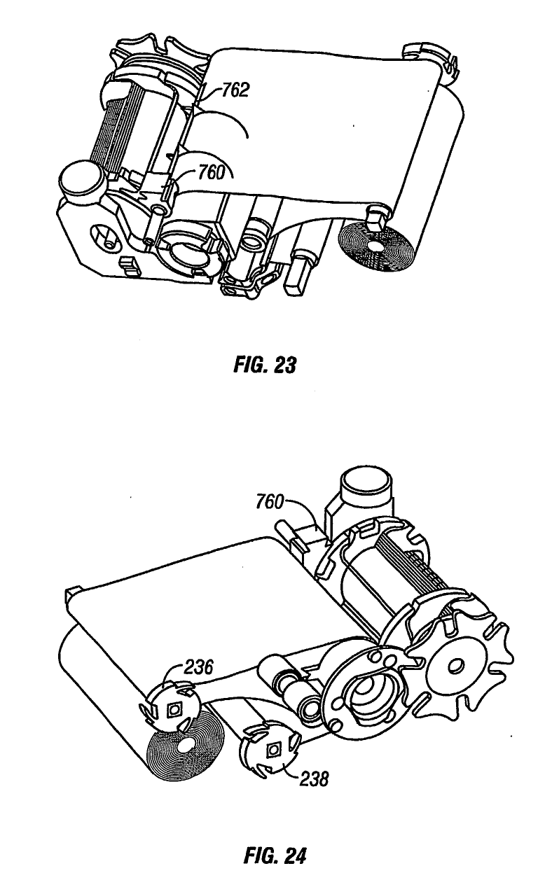

FIG. 22 shows one embodiment of the invention with the differential 740 in place to drive the take up spools 236 and 238. FIGS. 23 and 24 show close up views of one embodiment of the tape and spools according to the present invention. As seen in FIG. 23, a drive chuck 760 is used to drive a penetrating member 762.

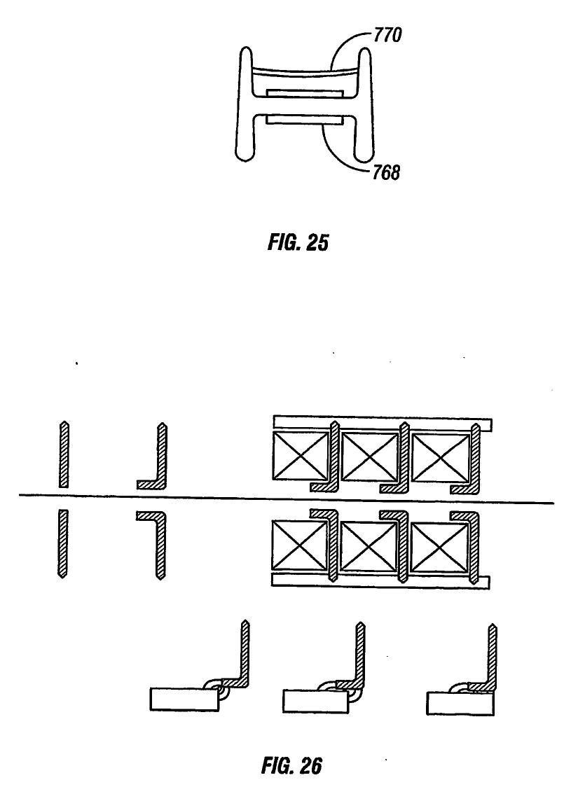

Referring now to FIG. 25, another embodiment of a tape take-up spool is described. A simple take-up spool arrangement is described that saves using multiple spools with friction clutches, or take-up idler rollers to maintain tension on two co-wound tape strips of unequal length.

In this embodiment, the technique of storing multiple penetrating members in a strip of foil tape is described. Each penetrating member is protected and maintained sterile by sealing it individually in a pouch created by bonding two strips of tape, upper and lower, together around the penetrating member. A penetrating member actuator then peels the tapes apart to expose individual penetrating members for loading into the launching mechanism. A problem arises when we decide how to store the used tape strips. To save space, and simplify the drive mechanism, it is desirable to wind both tape strips on a single spool. Because of stretch in the tapes, and sometimes because of changes in winding direction, the tapes are of different length. Separate take-up spools can be driven differentially, or with slip-clutches to accommodate differences in tape length. A single take-up spool may use a spring-loaded idler roller to maintain tension on both strips.

A simpler solution to this problem can be achieved by making the tape that is longer wider than the other tape strip, and slightly wider than the flanges of the take-up spool (see FIG. 25). The shorter strip 768 lays on the hub of the spool, and the longer strip 770, being wider, is supported by the flanges of the spool. Once excess slack is taken up in the longer tape, the increased tension will pull the tape down to some intermediate position between the spool flanges. Because the longer tape is winding on a larger diameter, the excess length is taken up. As the spool continues to turn and receive tape, the shorter narrow tape overlays the previous wraps of the longer tape. Tension in the shorter tape forces the longer tape down onto the hub of the spool so that most of the tape is stored as layer-on-layer of alternating short and long tape. In the top layer, the two tapes separate to make up the difference in length between the two tapes.

In a situation where an initial length difference between the two tapes is resolved as the tapes are used, the separation between the tapes will reduce until the tapes are winding essentially on top of each other. In one embodiment, a take-up spool arrangement is provided for storing tape strips of unequal length, maintaining tension on both tape strips, or eliminating mechanical components.

FIG. 26 shows configurations for holding penetrating members on a tape. Although the penetrating members are shown to have a L configuration, other shaped penetrating members such as but not limited to straight configurations may be used as well.

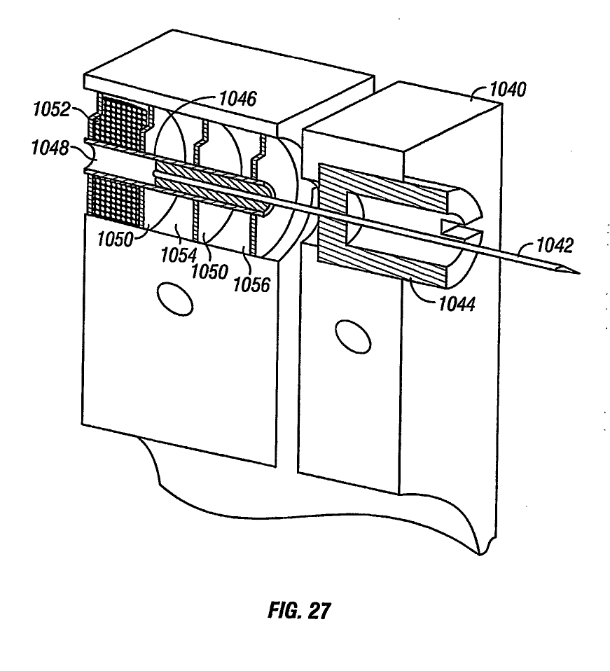

The present invention may be used with a variety of different penetrating member drivers. It is contemplated that these penetrating member drivers may be spring based, solenoid based, magnetic driver based, nanomuscle based, or based on any other mechanism useful in moving a penetrating member along a path into tissue. It should be noted that the present invention is not limited by the type of driver used with the penetrating member feed mechanism. One suitable penetrating member driver for use with the present invention is shown in FIG. 27. This is an embodiment of a solenoid type electromagnetic driver that is capable of driving an iron core or slug mounted to the penetrating member assembly using a direct current (DC) power supply. The electromagnetic driver includes a driver coil pack that is divided into three separate coils along the path of the penetrating member, two end coils and a middle coil. Direct current is alternated to the coils to advance and retract the penetrating member. Although the driver coil pack is shown with three coils, any suitable number of coils may be used, for example, 4, 5, 6, 7 or more coils may be used.

Referring to the embodiment of FIG. 27, the stationary iron housing 1040 may contain the driver coil pack with a first coil 1052 flanked by iron spacers 1050 which concentrate the magnetic flux at the inner diameter creating magnetic poles. The inner insulating housing 1048 isolates the penetrating member 1042 and iron core 1046 from the coils and provides a smooth, low friction guide surface. The penetrating member guide 1044 further centers the penetrating member 1042 and iron core 1046. The penetrating member 1042 is protracted and retracted by alternating the current between the first coil 1052, the middle coil, and the third coil to attract the iron core 1046. Reversing the coil sequence and attracting the core and penetrating member back into the housing retracts the penetrating member. The penetrating member guide 1044 also serves as a stop for the iron core 46 mounted to the penetrating member 1042.

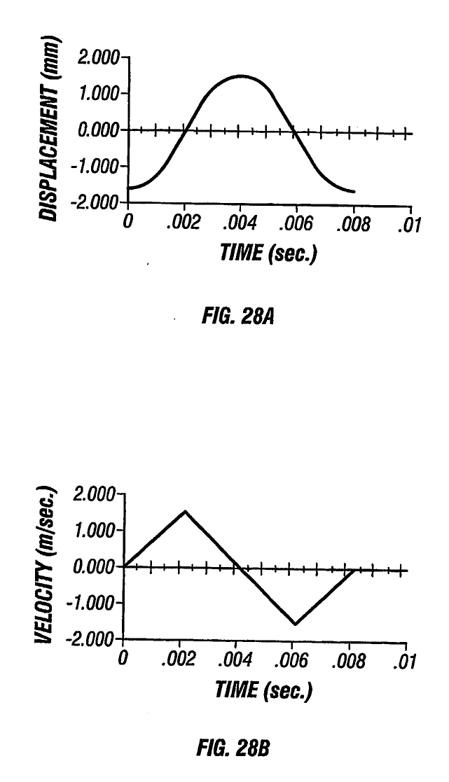

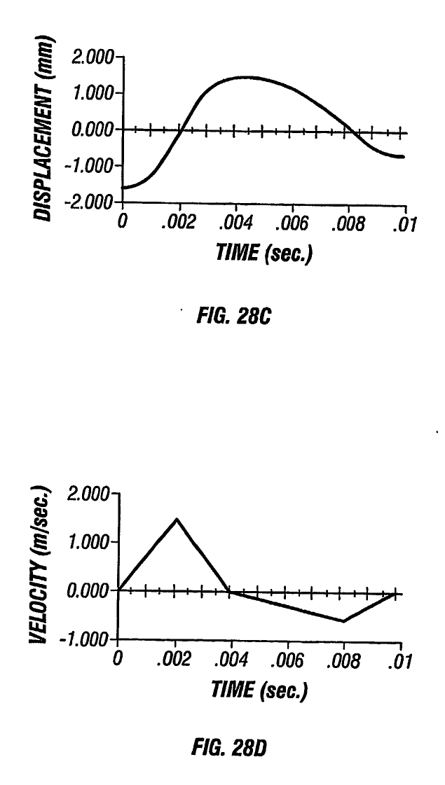

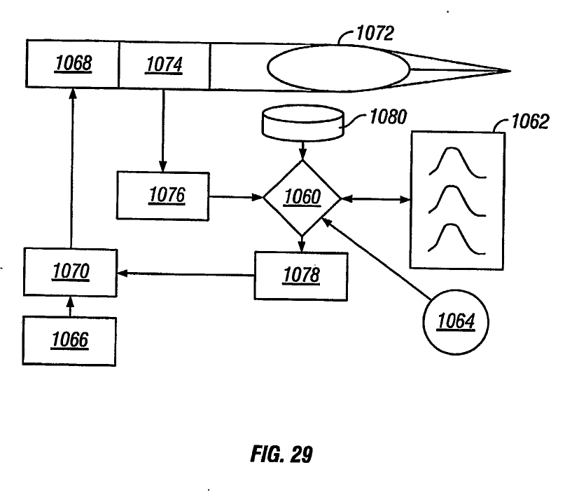

As discussed above, tissue penetration devices which employ spring or cam driving methods have a symmetrical or nearly symmetrical actuation displacement and velocity profiles on the advancement and retraction of the penetrating member as shown in FIGS. 28 and 29. In most of the available penetrating member devices, once the launch is initiated, the stored energy determines the velocity profile until the energy is dissipated. Controlling impact, retraction velocity, and dwell time of the penetrating member within the tissue can be useful in order to achieve a high success rate while accommodating variations in skin properties and minimize pain. Advantages can be achieved by taking into account of the fact that tissue dwell time is related to the amount of skin deformation as the penetrating member tries to puncture the surface of the skin and variance in skin deformation from patient to patient based on skin hydration.

In this embodiment, the ability to control velocity and depth of penetration may be achieved by use of a controllable force driver where feedback is an integral part of driver control. Such drivers can control either metal or polymeric penetrating members or any other type of tissue penetration element. The dynamic control of such a driver is illustrated in FIG. 28C which illustrates an embodiment of a controlled displacement profile and FIG. 28D which illustrates an embodiment of a the controlled velocity profile. These are compared to FIGS. 28A and 28B, which illustrate embodiments of displacement and velocity profiles, respectively, of a harmonic spring/mass powered driver. Reduced pain can be achieved by using impact velocities of greater than about 2 m/s entry of a tissue penetrating element, such as but not limited to a penetrating member, into tissue. Other suitable embodiments of the penetrating member driver are described in commonly assigned, copending U.S. patent application Ser. No. 10/127,395, (Attorney Docket No. 38187-2551) filed Apr. 19, 2002 and previously incorporated herein.

FIG. 29 illustrates the operation of a feedback loop using a processor 1060. The processor 1060 stores profiles 1062 in non-volatile memory. A user inputs information 1064 about the desired circumstances or parameters for a lancing event. The processor 1060 selects a driver profile 1062 from a set of alternative driver profiles that have been preprogrammed in the processor 1060 based on typical or desired tissue penetration device performance determined through testing at the factory or as programmed in by the operator. The processor 1060 may customize by either scaling or modifying the profile based on additional user input information 1064. Once the processor has chosen and customized the profile, the processor 1060 is ready to modulate the power from the power supply 1066 to the penetrating member driver 11068 through an amplifier 1070. The processor 1060 may measure the location of the penetrating member 1072 using a position sensing mechanism 1074 through an analog to digital converter 1076 linear encoder or other such transducer. Examples of position sensing mechanisms have been described in the embodiments above and may be found in the specification for commonly assigned, copending U.S. patent application Ser. No. 10/127,395, (Attorney Docket No. 38187-2551) filed Apr. 19, 2002 and previously incorporated herein. The processor 1060 calculates the movement of the penetrating member by comparing the actual profile of the penetrating member to the predetermined profile. The processor 1060 modulates the power to the penetrating member driver 11068 through a signal generator 1078, which may control the amplifier 1070 so that the actual velocity profile of the penetrating member does not exceed the predetermined profile by more than a preset error limit. The error limit is the accuracy in the control of the penetrating member. In one embodiment, the average penetration velocity of the penetrating member from the point of contact with the skin to the point of maximum penetration may be about 2.0 to about 10.0 m/s, specifically, about 3.8 to about 4.2 m/s. In another embodiment, the average penetration velocity of the penetrating member may be from about 2 to about 8 meters per second, specifically, about 2 to about 4 m/s.

After the lancing event, the processor 1060 can allow the user to rank the results of the lancing event. The processor 1060 stores these results and constructs a database 1080 for the individual user. Using the database 1080, the processor 1060 calculates the profile traits such as but not limited to degree of painlessness, success rate, and blood volume for various profiles 1062 depending on user input information 1064 to optimize the profile to the individual user for subsequent lancing cycles. These profile traits depend on the characteristic phases of penetrating member advancement and retraction. The processor 1060 uses these calculations to optimize profiles 1062 for each user. In addition to user input information 1064, an internal clock allows storage in the database 1080 of information such as but not limited to the time of day to generate a time stamp for the lancing event and the time between lancing events to anticipate the user's diurnal needs. The database stores information and statistics for each user and each profile that particular user uses.

In addition to varying the profiles, the processor 1060 can be used to calculate the appropriate penetrating member diameter and geometry necessary to realize the blood volume desired by the user. For example, if the user desires about 1-5 microliter volume of blood, the processor 1060 may select a 200 micron diameter penetrating member to achieve these results. For each class of penetrating member, both diameter and penetrating member tip geometry, is stored in the processor 1060 to correspond with upper and lower limits of attainable blood volume based on the predetermined displacement and velocity profiles.

The lancing device is capable of prompting the user for information at the beginning and the end of the lancing event to more adequately suit the user. The goal is to either change to a different profile or modify an existing profile. Once the profile is set, the force driving the penetrating member is varied during advancement and retraction to follow the profile. The method of lancing using the lancing device comprises selecting a profile, lancing according to the selected profile, determining lancing profile traits for each characteristic phase of the lancing cycle, and optimizing profile traits for subsequent lancing events.

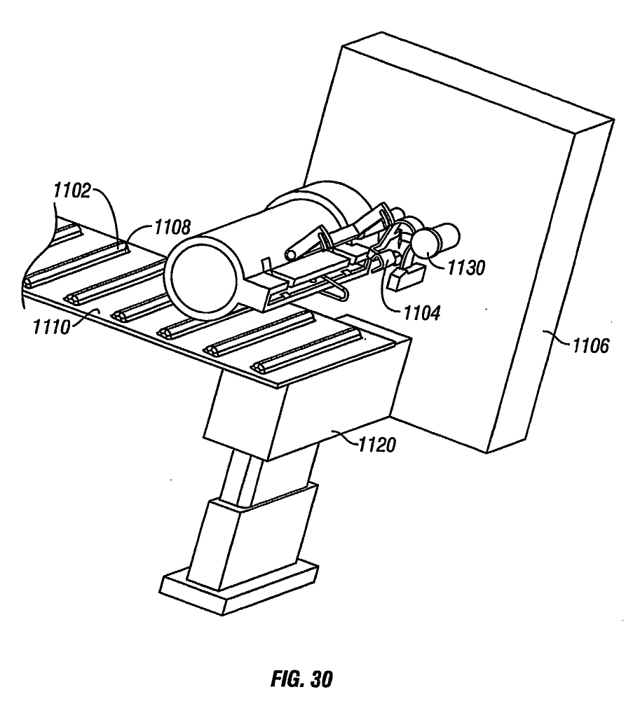

Referring now to FIG. 30, one embodiment of a feed system or mechanism 1100 for use with a penetrating member driver will be described. As seen in FIG. 30, a plurality of penetrating members 1102 may be arranged in an array configuration and fed towards a penetrating member coupler 1104 of a penetrating member driver 1106. In this embodiment, the penetrating members 1102, or the housings that they may be mounted in, may be serially interconnected. The precise dimensions of the penetrating members 1102, number of penetrating members, and configuration of the array are a function of several variables including but not limited to the amount of body fluid to be sampled, depth of penetrating member penetration, how often the body fluid is sampled per day, the type of penetrating member driver used, and the like. The penetrating member may feature shape variation or surface treatment on a non-cutting end of the penetrating member to enable or enhance handling of the penetrating member. In one embodiment, the penetrating member may have a diameter of about 317 microns. The length may be between about 10-20 mm, and may be 11.3 mm. In one embodiment, the bent leg portion may have a length between 1-3 mm, and may be 2.65 mm. About 3-5 mm of the penetrating member may be coated, such as but not limited to about 4 mm. The penetrating member may be made of a variety of materials such as but not limited to 304 stainless steel, steel, steel alloys, shape memory materials, ceramic, other metal alloys, or other materials useful for piercing tissue.

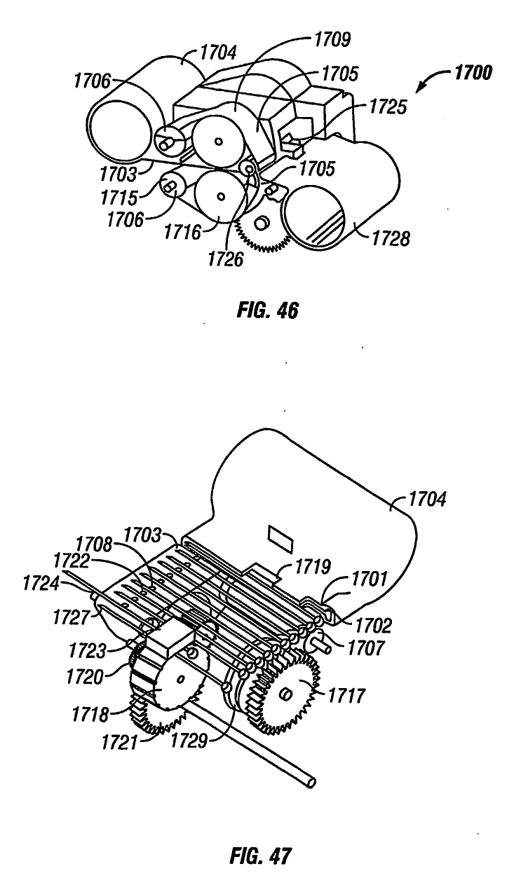

In this particular embodiment, penetrating members 1102 are packaged in a sterile environment provided by penetrating member enclosures 108 such as but not limited to blisters or pouches on a tape 1110. The tape 1110 may comprise of a base layer and a cover layer, bonded together to create a closed cavity or sterile environment that surrounds each penetrating member. During manufacture of this particular embodiment, the penetrating members 1102 may be sterilized in their blisters and remain sterile until use. As the penetrating members are used, the tape of penetrating members may be advanced by a transport device such as but not limited to a roller or cogs (such as but not limited to in FIGS. 46 and 47) that roll the tape past the penetrating member driver 1106. It is noted that the rollers or cogs may be substituted by any mechanism useful in transporting a tape of penetrating members, a bandolier of penetrating members, or a plurality of penetrating members otherwise arranged in an array, towards a penetrating member driver or penetrating member coupler. For example, a conveyor belt may be employed or a spring based urging device in a magazine/cartridge for advancing the penetrating members.

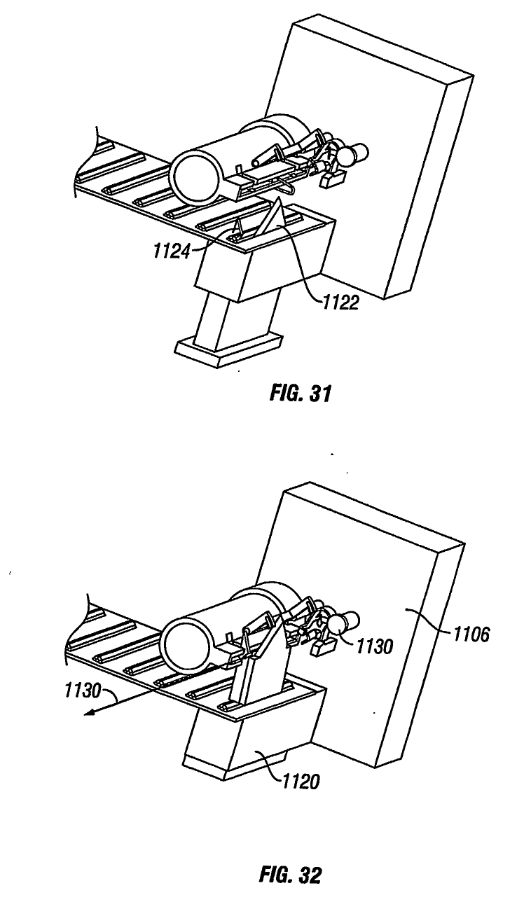

In FIG. 31, a penetrating member release device 120 is deployed to remove a penetrating member 1102 from the sealed enclosure 108 and move the penetrating member towards the penetrating member coupler 1104. In this particular embodiment, the release device 120 includes a first knife blade 122 that moves through the tape 1110 to slice the base tape and enclosure 108 along the length of the penetrating member 1102. Secondary blades 124 may produce perpendicular cuts so that a flap is created from the blister or enclosure 108. It should be understood that the secondary blades 124 may cut the enclosure at other angles besides perpendicular and still release the penetrating member 1102. In other embodiments, secondary blades 24 may be of different shapes or not included at all. In the present embodiment, the blades 122 and 124 join to form a cradle or other configuration that captures and lifts the penetrating member 1102 into the penetrating member coupler or penetrating member coupler 1104. Of course, it should be noted that at least a portion of the cradle may be formed by non-blade portions of the release device, so long as these portions can provide sufficient support to engage and move the penetrating member 1102. In the preferred embodiment, flaps are created and folded open.

Referring now to the embodiment of FIG. 32, the release device 1120 is shown fully extended and the penetrating member is positioned in the open penetrating member coupler 1104. A portion of the lifting mechanism (not shown) of the release device 1120 lifts the penetrating member coupler 1104 off of its retaining catch allowing it to move toward the penetrating member coupler 1102, seating the penetrating member in the coupler as a retaining spring in the coupler closes to capture the penetrating member 1102. It should be understood that a variety of different penetrating member couplers may be used and FIG. 32 is merely illustrative of one suitable embodiment for use with a penetrating member driver 1106 and feed mechanism 1100. Any device useful for engaging a penetrating member in a manner sufficient to drive a penetrating member into tissue may be used. The device or coupler may be combined with any supporting elements (if necessary) to stablize the penetrating member during its actuation. The penetrating member 1102 when coupled to the penetrating member coupler 1104 may then be advanced into the patient as indicated by arrow 1130. The blades 1122 and 1124 may, but does not necessarily, remain in a fully extended position to support and guide the penetrating member. It should be understood that the blades 1122 and 1124 may also be retracted, in which case the penetrating member 1102 may be guided by some other device or simply be advanced by the penetrating member driver without additional support. Any mechanism useful for guiding a penetrating member or an elongate member such as but not limited to a slot, groove, two aligned openings, or the like may be used.

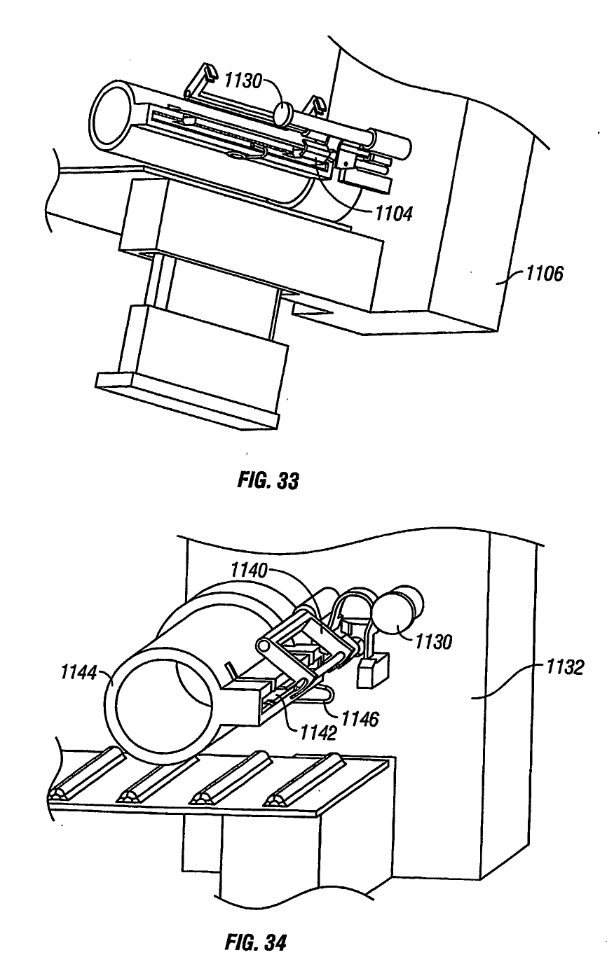

In this embodiment during the removal of the used penetrating member, the retractor 1130 slides toward the base plate, forcing ears on the retaining spring over a wedge feature on the base plate, opening the penetrating member coupler 1104 or drive chuck, releasing the used penetrating member. The lance coupler 1104 in this embodiment is designed to move forward and capture the fresh penetrating member. When the knife blades 122 and 124 are in their parked position, the retractor 130 is extended to allow free motion of the actuator, and the penetrating member 1102 is ready for launch. After use, the penetrating member 1102 may park in the position shown in FIG. 33.

During the penetrating member disposal cycle shown in FIG. 34, the retractor 130 may draw the penetrating member coupler 1104 toward the base plate 132 until the clamp spring is forced open and the coupler 1104 latches in its open position. The penetrating member unloading device 140, which in this embodiment may be a scoop, rotates to capture the penetrating member 1102 before the penetrating member coupler 1104 releases it, and then forces the penetrating member 1102 into the opening 1142 of the collection bin 1144 past the retainer spring 1146. The penetrating member unloading device 1140 then returns to its parked position and the penetrating member driver 1106 and feed mechanism 1100 is ready to begin the next cycle. It should be noted that the scoop may be replaced by any mechanism useful for engaging the penetrating member and moving it from the penetrating member coupler 1104 to the collection bin 1144. For example and not by way of limitation,

One advantage of this particular embodiment of the present invention is that the mechanism that handles the used penetrating members, unloading device 1140, may be isolated from the mechanism (release device 1120) that loads the sterile penetrating members. This reduces the chance of contamination of the new penetrating members by used penetrating members having fluids or other contaminants from the tissue. Additionally, in this embodiment, the functions of the penetrating member release device and the penetrating member load device are combined into one mechanism (device 1120).

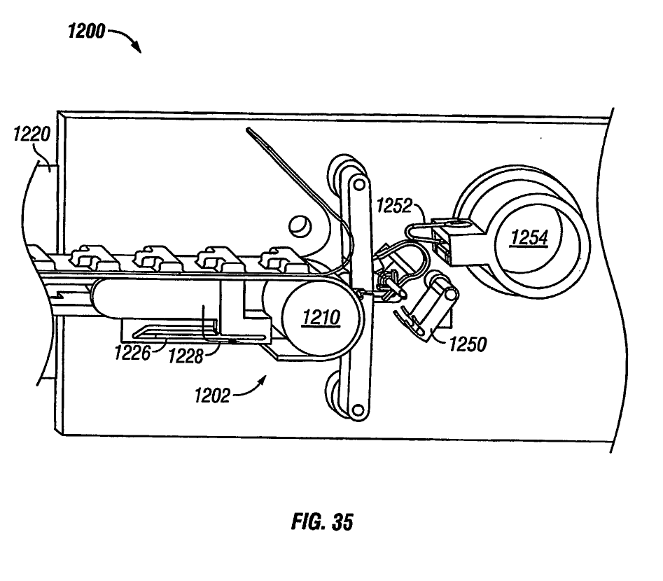

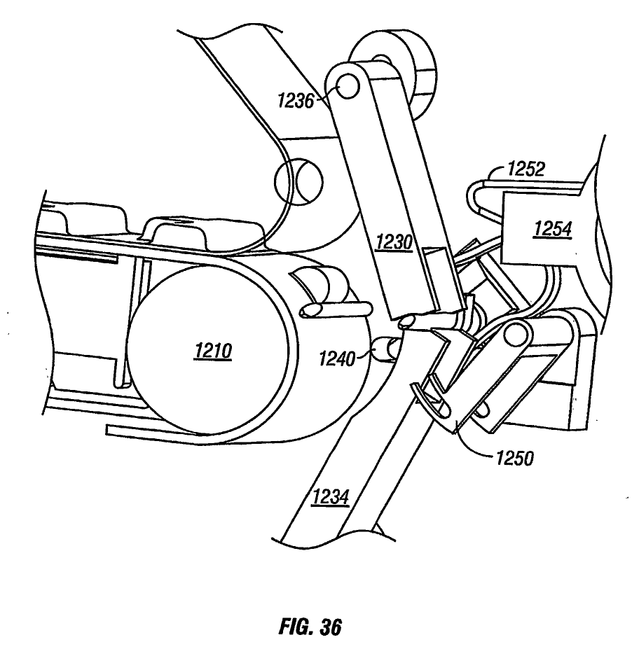

Referring now to FIG. 35, another embodiment of a feed mechanism 1200 for use in a tissue penetrating system will be described. Again, sterile penetrating members 1102 may be packaged in penetrating member enclosures such as but not limited to, but no limited to, blisters or pouches on a tape 1110. The penetrating members 1102 may be individually packaged in each blister or pouch or packaged in groups. The tape 1110 in this embodiment may also comprises of a base layer and a cover layer bonded together in a sealing manner about the penetrating members 1102. The tape 1110 may be made of a variety of materials including but not limited to aluminum foil with heat activated bonding layer, metallic foil, aluminum foil, paper, polymeric material, or laminates combining any of the above. A suitable material includes aluminum foil with heat activated bending layer. The two layers may be peeled apart by the release device 1202, releasing the penetrating members 1102 one by one. To assist in the penetrating member transfer, the base layer of tape 1110 in this embodiment may be, but is not necessarily, adhesively coated to retain the penetrating member 1102 once the cover layer is removed. To further assist in penetrating member transfer, a blister, or pusher bar, is formed in the base to help in lifting the penetrating member off of the tape. In this present embodiment, separate mechanisms may be used to handle the clean penetrating members 1102 and used penetrating members to reduce the risk of contamination. For example in the embodiment of FIG. 35, the roller 1210 may act as both a release device and a transport device as it advances the tape 1110. The roller 1210 may peel the tape apart as each penetrating member nears the penetrating member driver 1212. A separate mechanism may be used for removing used penetrating members.

In the present embodiment, the penetrating member transport is driven by a cam plate 1220 at the back of the mechanism (and as more clearly shown in FIG. 37). Although a cam plate is used, it should be understood that other devices such as but not limited to gears, belts, push rods, or other devices may be used separately or in combination with the cam plate for actuation and/or sequencing. The cam plate 1220 drives a cog 1222 that engages the blister or other feature in the base layer of tape 1110. In this embodiment, the cam plate 1220 acts as a sequencing cam plate to help the various parts coordinate their actions. Tension is maintained in take-up rollers 1210 for the cover and base layer (for ease of illustration, the roller for the cover layer is not shown). Although not necessarily the case, in this embodiment the tension may provide most of the peeling energy, allowing the cog 1222 to control speed and position of the penetrating member. The rollers 1210 may be idlers or may be actively driven to assist in the pealing process. As the cam plate moves plate 1220 from left to right, the cog 1222 moves a penetrating member 1102 toward the transfer point. When a fresh penetrating member 1102 is in position, the cog 1222 is moved out of engagement with the tape by its cam surface 1226 shown below the cog in FIG. 35 and as indicated by arrow 1228. The cog 1222 continues to travel to the right, driven by the motion of the cam plate 1220.

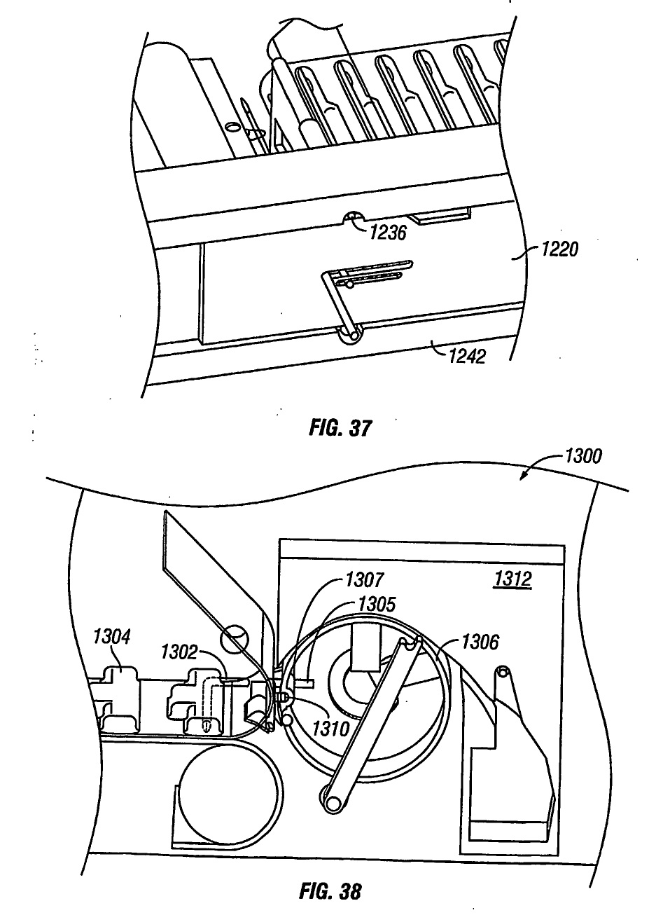

Referring now to FIG. 36, as a fresh penetrating member moves toward the transfer point, it passes under the upper transfer fork 1230 of the penetrating member loading device 1232. The upper fork 1230 provides an upper surface to grip the penetrating member 1102. The upper fork 1230 may be, but is not necessarily, spring loaded so it can move to allow the penetrating member 1102 to pass beneath it. When the penetrating member 1102 reaches the lower transfer fork 1234, the penetrating member 1102 is blocked from further motion by the fork, and enough clearance is provided by the upper fork pivot pin 1236 riding on a lifting surface on the cam plate 1220 (see FIG. 37) to allow the upper fork 1230 to close behind the penetrating member. Any further motion by the tape will cause the blister on the base tape to slide under the penetrating member and push the penetrating member away from the adhesive on the base tape. An appropriate shape for the leading edge of the blister will be determined to promote this transfer with minimum bending stress in the penetrating member.

Continued motion of the cam plate 1220 brings the end of a clearance slot (or other drive surface) into contact with a pin 1238 attached to the lower transfer fork. The pin may be guided by a slot 1240 in the base plate 1242 of the mechanism. The pivots of the transfer forks move in slots, allowing the penetrating member path to be determined by the base plate slot. In this embodiment, the upper transfer fork 1230 is spring loaded against the lower fork 1234 to both clamp or grip the penetrating member during most of the travel, and hold the upper fork drive pin 1236 against the lower fork drive pin so the forks move together. The rolling action of the forks 1230 and 1234 caused by their pivots moving in slots acts to provide a closed containment area for the penetrating member 1102 when it is acquired, and a guide slot for the penetrating member when it is inserted into the penetrating member coupler 1104.

During the unloading cycle, the penetrating member coupler 1104 may be pulled against a tapered surface on the base plate that wedges the clamp spring (purple) open (mechanism not shown at this time). The penetrating member coupler 1104 is held in the open position by a catch (not shown) on the base plate 1242. By contrast, during the loading cycle, the transfer forks 1230 and 1234 place the penetrating member 1102 into the open penetrating member coupler 1104 when the forks reach the end of their travel and release the catch allowing the penetrating member coupler to move off of the wedge and toward the penetrating member, seating the penetrating member into the penetrating member coupler as the clamp spring captures the penetrating member. The cam plate 1220 then returns slightly to pull the drive forks clear of the penetrating member coupler 1104 to allow free operation of the penetrating member coupler.

In the present embodiment, a rack segment (not shown) on the cam plate 1220 engages a drive gear (not shown) on the penetrating member unloading device 1250 which may be a extractor fork as more clearly seen in FIG. 36. The drive gear drives the unloading device 1250 through a one-way clutch that allows the gear to free wheel during the left-to-right motion of the cam (FIG. 36). As the cam plate 1220 reverses, a retractor (not shown) draws the penetrating member coupler 1104 onto the opening wedge and latches it as the drive gear rotates the unloading device 1250 to capture the penetrating member 1102 and force it past a retainer spring 1252 into the collection bin or storage canister 1254 for used penetrating members. When this action is complete, the drive gear falls off of the end of the rack segment and a return spring returns the penetrating member unloading device 1250 to its starting position (FIGS. 36 and 37).

As the cam plate 1220 moves to the left end of its travel, returning to its starting position, the cog cam 1222 is drawn up a ramp in its cam groove, and drops down into the starting position in the groove ready to lift and index the next penetrating member into position. As previously mentioned, a combination of cam plates and gears, slots, belts, pushbars, or other devices may also be used. Some advantages of this particular embodiment are that its penetrating members are stored in a sterile environment until just before use, separate elements of the mechanism handle clean and used penetrating members, and waste may be contained within the device.

In a still further embodiment of the present invention, a feed mechanism 1300 for use with L or other shaped penetrating members will now be described. As in previous embodiments, sterile penetrating members 302 may be packaged in penetrating member enclosures 304 such as but not limited to, but not limited to, blisters, pouches on a tape, tightly sealed between the tapes without air pockets, or other sealing configurations protecting against penetrating member contamination. The present invention is particularly well suited for use with an L-shaped or bent penetrating member because one leg 305 of the penetrating member may be slidably engaged by a penetrating member loading device 1306. It should be understood, of course, that other penetrating members having various other shapes may also be used as long as they have some type of abutment, protrusion, attachment, or shaped configuration that allows the penetrating member loading device 1306 to engage the penetrating member.

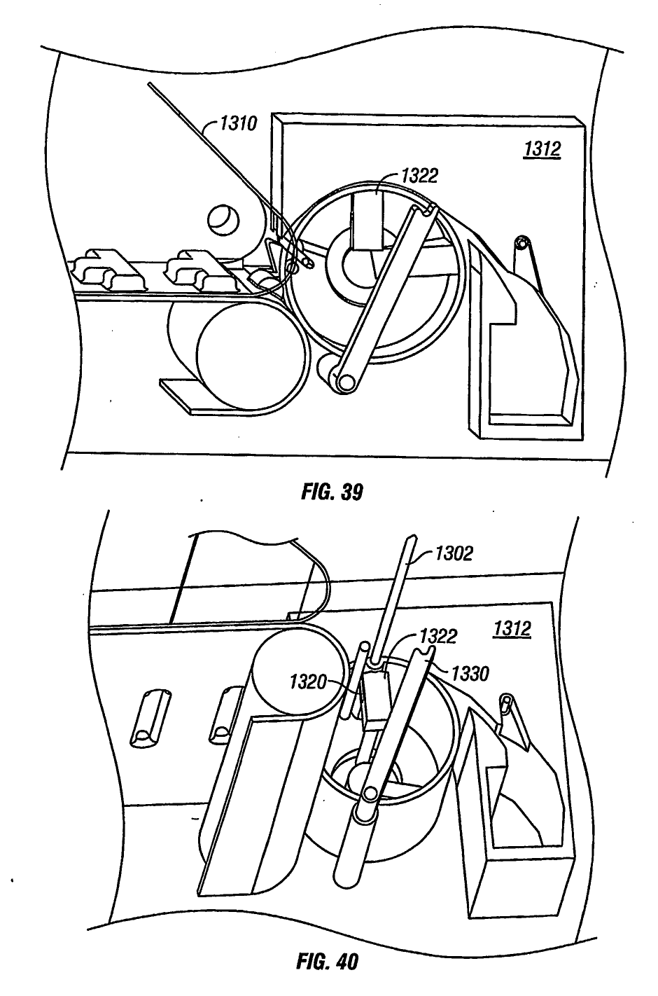

Referring now to FIG. 38, feed mechanism 1300 is shown having a penetrating member 1302 partially inserted into the penetrating member loading device 1306 which in this embodiment may be a transfer drum at the end of the tape peel action. The drum may have an opening 1307 for receiving the penetrating member 1302. Any mechanism having a contour or opening for engaging or constraining a penetrating member may be used. As seen in FIG. 38, the peeling action of the tape has progressed from the end of the bent portion of the penetrating member 1306 toward the long “body” portion. The body of the penetrating member 1302 is held by the tape as the bent leg 1305 is inserted into the penetrating member loading device 1306 and guide surfaces.

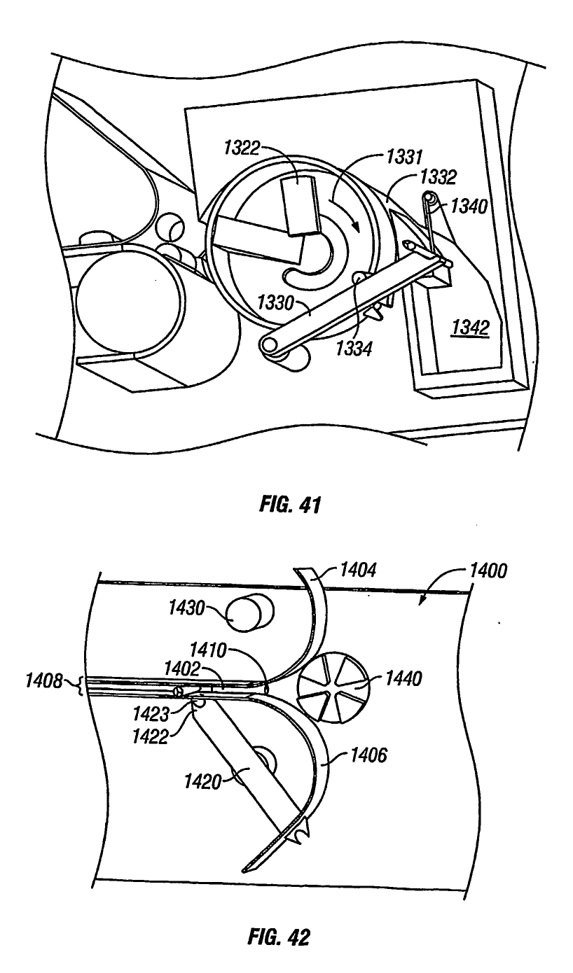

FIG. 39 shows the loading device 1306 as it begins to rotate. The penetrating member 1302 is lifted into a guide channel 1310 that completes the insertion process leaving the penetrating member 1302 completely contained by the guide channel 1310 and the fixed housing 1312 in which the penetrating member loading device 1306 rotates. Sides of the groove at the bent end 1305 of the penetrating member prevent the penetrating member from rotating and constrain the bent portion of the penetrating member to a radial position relative to the loading device 1306. As the loading device 1306 reaches the firing or launch position, the bent portion 1305 of the penetrating member 1302 enters a slot 1320 in the drive coupler 1322 attached to the penetrating member driver. The slot 1320 is more clearly shown in FIG. 40.

In FIG. 40, the drive coupler 1322 and penetrating member 1302 are shown in the fully extended position. The penetrating member 1302 has been advanced by the driver coupler 1322 which is attached to the penetrating member driver. The penetrating member loading device 1306 may be locked in the launch position that guides the tip of the penetrating member 1302 through an aperture in the outer housing (not shown for ease of illustration).

Referring now to FIG. 41, the penetrating member 1302 is shown being lifted from the transfer drum by the extractor bar 1330. In this embodiment, the transfer acts as a penetrating member loading device 1306 and as part of the penetrating member unloading device. As the transfer drum rotates away from the firing position as indicated by arrow 1331, the penetrating member 1302 engages the extractor bar 1330 that begins to lift the penetrating member from the guide groove 1332 (see FIG. 41) defined between the transfer drum and the housing 1312. When the penetrating member 1302 clears the guide groove 1332, the extractor bar 1330 continues to be driven by a pin 1334 on the transfer drum. The extractor bar 1330 forces the penetrating member 1302 past a spring 1340 that retains the penetrating member in the used penetrating member bin 1342. FIG. 41 represents the maximum rotation of the transfer drum in this embodiment. As the drum rotates back to the loading position, the extractor bar 1330, driven by its return spring (not shown), slides the used penetrating member up the retainer spring until the penetrating member falls off of the extractor bar and is captured in the used penetrating member bin 1342 portion of the housing.

In the present embodiment, the desired throw of the penetrating member driver and the length of guide groove 1332 necessary for accurate penetrating member motion determines the length of the transfer drum. Additional penetrating member length may be added to provide a standoff distance between the drum and the aperture in the outer housing to isolate the drum from any contamination from blood entering the aperture. An additional shield consisting of an internal surface with a hole of similar size as the aperture may be provided along the path of the penetrating member. The extractor bar 1330 can be protected from contamination, but it should be understood that the extractor does not contact the penetrating member until after the penetrating member has been used.

Alternatively in this present embodiment, the active penetrating member may be constrained from all motion except along its longitudinal axis and the actuator drive coupling is fixed while the transfer drum moves the penetrating member. An alternative design would consist of multiple actuators, such as but not limited to a high-speed solenoid for actuation and depth control, and a low speed actuator such as but not limited to a motor, nanomuscle, or any other controllable mechansim for retraction. The drive coupling 1322 could be eliminated and replaced with a simple plunger attached to the solenoid that pushes on the end of the penetrating member for actuation, and then retracts to its rest position. The retraction mechanism may be connected to an “L” shaped paddle that would act on the under side (opposite the drive solenoid) of the penetrating member drive leg. The retraction mechanism would be deployed before the solenoid-driven actuation stroke, and could act as a travel stop. After the actuation cycle and any dwell time, the retraction drive would retract the paddle to bring the penetrating member to its “park” position.

Referring now to FIG. 42, a still further embodiment of a feed mechanism 1400 will now be described. FIG. 42 shows the basic mechanism with a penetrating member 1402 held between two layers of tape 1404 and 1406. For simplicity and ease of illustration, the tape 1408 is drawn as two flat layers 1404 and 1406 separated by the penetrating member 1402. In actual implementation, the tape 1408 may be, but is not necessarily, formed around the penetrating member 1402 and sealed around the perimeter to form a separate pouch around each penetrating member. The penetrating member 1402 may consist of a straight wire with one end ground to a sharp point with adjacent cutting edges, and with the other end 1410 bent into a “L” shape with the leg facing forward as the penetrating member feeds. A penetrating member release device 1420 may be a rotatable member such as but not limited to, but not limited to, a sharpened blade 1422 that is integrated into a roller around which the lower tape is wrapped. It should be understood that the sharpened blade may have a variety of shapes (not shown) and the FIG. 42 shows only one possible embodiment. The upper tape wraps around an upper roller (not shown but would follow the curvature of the upper tape), and together the rollers peel the tape halves 404 and 406 apart releasing the penetrating member 1402.

In this embodiment, FIG. 42 shows the blade 1422 piercing the tape to capture the penetrating member 1402 before the tape releases it. As seen in the figure, the penetrating member may be captured while still in contact with the upper and lower layers of the tape 1408. Alternatively, the penetrating member 1402 may be captured with one of the layers (upper or lower) released from contact with penetrating member 1402. The penetrating member 1402 may be advanced until it is fully engaged by the penetrating member coupler 1440. In FIG. 42, the blade has a carve out 1423 to partially encircle or cradle the penetrating member 1402. As the rotating member turns, the penetrating member 1402 is lifted from the lower tape by the blade 1422. The upper roller 1430 is resilient, or relieved to allow this motion. The penetrating member leg 1410 is progressively engaging the penetrating member coupler 1440.

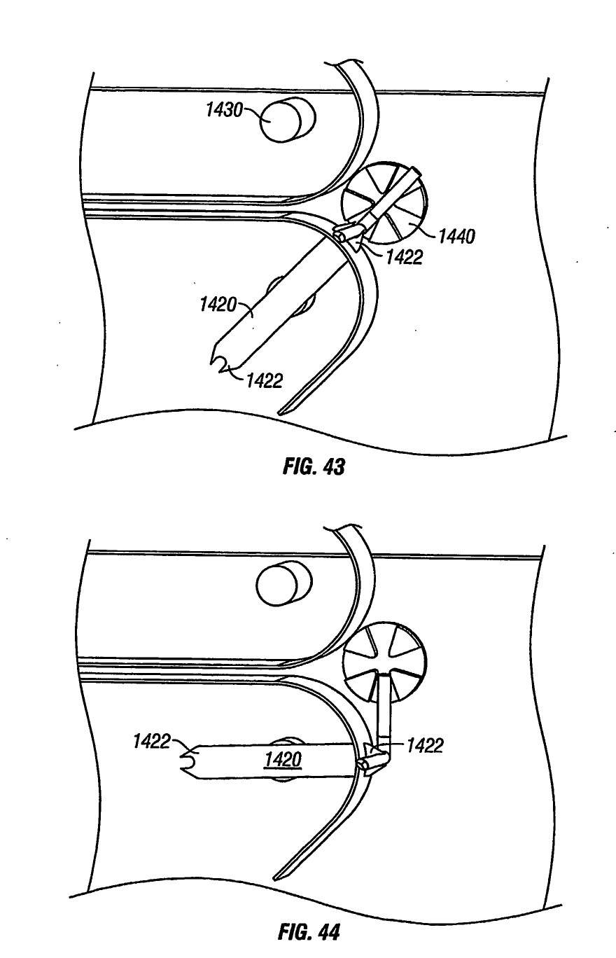

FIG. 43 shows the penetrating member 1402 in the launch position for this embodiment of the present invention. A fixed guide surface (see FIG. 45) captures the penetrating member in the blade groove as it leaves the upper tape. The penetrating member is guided during actuation by the grooves in the two blades (upper blade not shown) and the fixed guide surface. The top of the penetrating member coupler 1440 has been removed to show the groove detail, in a functional unit, the grooves 1442 are tapered ports in the penetrating member coupler 1440 so the penetrating member leg 1410 is captured for both extension and retraction. The penetrating member coupler 1440 has been rotated by the penetrating member head and the relative motion of the blade and the penetrating member coupler. In the present embodiment, a small amount of friction in the penetrating member coupler 1440 may be desirable to hold it steady as the penetrating member leg engages the coupler. During penetrating member actuation, the penetrating member coupler 1440 will be advanced toward the target tissue and in doing so, advance the penetrating member 1402 with it.

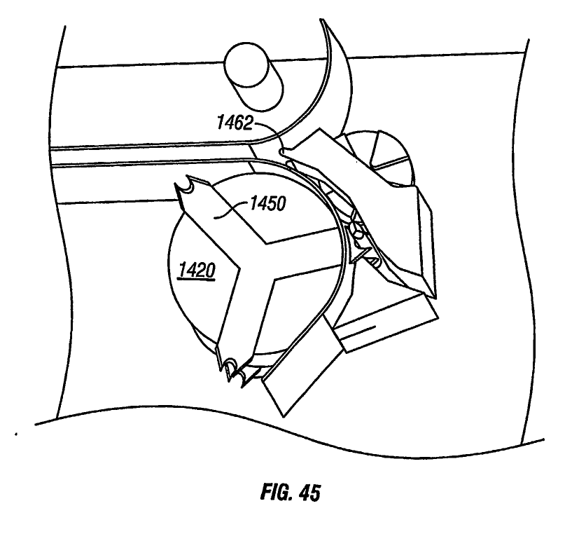

Referring now to FIG. 44, after the lancing is completed, the penetrating member 1402 is unloaded from the penetrating member coupler 1440. At this point, the fixed guide surface can branch away from the circular path of the blade and carry the used penetrating member into a waste bin. Alternatively if the fixed guide surfaces are not used or are shaped differently, as the blade 1422 rotates, the penetrating member 1402 will be carried to a point where it will be released from the penetrating member coupler and gravity will pull it from the slot on the blade 1422, depositing the penetrating member in storage bin or container. It should be understood that in some embodiments the system is not gravity dependent and that the penetrating member is held at substantially all times during transport and actuation.

Referring now to FIG. 45, another embodiment of the mechanism 1400 is shown with various changes and details added. FIG. 45 shows that the rotating member 1420 may include a three-armed piercing blade 1450 that may allow a reduced penetrating member pitch on the tape. The lower tape peeling roller of rotatable member 1420 is shown. As seen in FIG. 45, piercing blades may be located on both sides of the rotatable member 1420. The fixed guide surfaces 1460 are shown with the penetrating member 1402 at the point of transfer into the discharge slot. Two protrusions 1462 on the upper guide surface press into the upper tape to separate the penetrating member from the upper tape layer 1406. The upper peeling roller may be resilient, or relieved to allow this action.

Referring now to FIG. 46, a still further embodiment of a penetrating member loading and unloading assembly will be shown. FIG. 46 shows a perspective view of the assembly 1700. In this embodiment, each carrier tape may have a leader 1705 of un-bonded tape with a take-up spool 1706 attached. During loading, each leader is threaded into the mechanism and the take-up spool is engaged with the take-up drive 1707 (FIG. 21). The tape may be perforated with drive holes 1708 (FIG. 47) between the penetrating members. During loading, drive cogs 1709 may engage the drive holes in the tape to ensure positive positioning and for indexing of the penetrating members. Thus the tape may be both for sterility purposes and indexing purposes. Referring to the embodiment of FIG. 46, the tape can be seen passing between drive cogs 1715 and 1716, and separated by peel rollers 1725 and 1726. The tape then re-engages the drive cog as it wraps over the top of the cog and passes on to the take-up spool 1706. The secondary engagement of the tape allows the drive cog to produce force in the tape to break the adhesive and release the penetrating members. Moderate tension sufficient to keep the tape on the drive cogs is supplied by the take-up spool 1706, which is overdriven by a traction belt 1729 that slips to allow for a varying radius as the tape builds on the spool.

In the present embodiment, the two drive cog-wheels 1715 and 1716 are synchronized through gear 1717 and a mating gear (not shown) in the upper tape drive. A penetrating member guide wheel 1718 is synchronized with the tape drive cogs through gears 1719 and 1720, and thumbwheel 1721. As the thumbwheel 1721 is rotated, the drive cogs 1715 and 1716 rotate together to move the carrier tape and penetrating members into the next actuation position penetrating member 1722, still captured between the drive tapes, is picked up by the penetrating member guide wheel 1718, and captured in its guide slot by a fixed guide surface 1723 penetrating member 1724 is carried into the launch position by the rotation of the penetrating member guide wheel. As penetrating member 1724 moves from the staging position to the launch position peel rollers 1725 and 1726 separate the tapes fracturing the protective adhesive around the penetrating member. When it reaches the launch position, penetrating member 1724 may be, but is not necessarily, completely free of the carrier tapes, and fully supported by the penetrating member guide wheel 1718 and guide surface 1723. The spent penetrating member 1727 is carried into a collection cassette 1728 that may be, but is not necessarily, identical to the supply cassette 1704. The cassettes are shown with ends removed so used penetrating members can be seen. In all three figures, the active penetrating member 1724 is shown in the fully extended position, at the end of the penetration stroke, and just before retraction to the home position.

Referring to the embodiment of FIG. 47, completed penetrating members 1701 with drive heads 1702 are assembled side by side on a carrier tape 1703 of paper, plastic, or some other suitable material. A second carrier tape is assembled on top of the penetrating members so that the penetrating members are sandwiched between two layers of carrier tape. All of the components are held together with an adhesive that also maintains the sterility of the penetrating member tips. The assembly of penetrating members and carrier tapes is coiled and inserted into a cassette 1704 for transportation and long-term storage. The cassette and penetrating member/tape assembly may be sterilized at this point. A one-way clutch or ratchet mechanism (not shown) may be used to ensure that penetrating members travel in only one direction. An escapement mechanism (not shown) may be used to ensure that only one penetrating member is used per actuation cycle, and that each penetrating member is used only once.

The penetrating member actuation/advance cycle may be repeated until all of the penetrating members are used. At that point all of the penetrating members may be contained in the collection canister 1728, the supply canister 1704 may be empty, and the protective tape may be rolled up on the take-up spools 1706. In this embodiment, the repeating launcher is reloaded by lifting the hinged tape drive frame that rotates roughly six degrees about a pivot near the mesh line of the drive gears 1717. This ensures that the gears stay in mesh so the synchronization of the drive cogs 1715 and 1716 is not lost. With the tape drive frame raised, the tapes and take-up spools can be unwrapped from the drive cogs 1715 and 1716, and discarded. The used penetrating member cassette 1728 is removed and discarded, and the empty supply cassette 1704 is removed and inserted as the new used penetrating member cassette. In this embodiment, a fresh package with sterile supply cassette is opened; the cassette is placed into the launcher, the tape tails are threaded between the drive cogs 1715 and 1716, around the peeling rollers 1725 and 1726, over the cog pins 1709 on the back of the drive cog, and the take-up spools are dropped into cradles and engaged with the take-up mechanism 1707 through a drive tang on the end of the take-up spool.

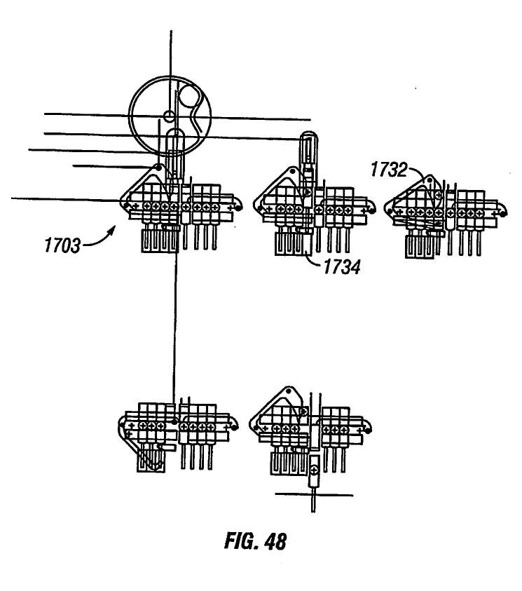

Referring to the embodiment of FIG. 48, this mechanism 1730 stores fresh penetrating members in a stack moving from left to right. A gate 1732 separates the next penetrating member to be used from the stack and advances it toward the launching mechanism, stripping the protective cap 1734 from the penetrating member. The penetrating member is released into the actuation mechanism for actuation. The used penetrating member is then pushed into the “used penetrating member” stack by the loading mechanism as it loads the next penetrating member. It should be understood that any mechanism useful for pulling off, pushing away from, slicing off, carving off, or otherwise removing the protective cap or sterility enclosure 1734 may be used. The protective cap 1734 may also be any device such as but not limited to a blister pack, fracturable or hinged protective cap or sleeve, useful for maintaining at least the tip of the penetrating member in a sterile condition prior to lancing.

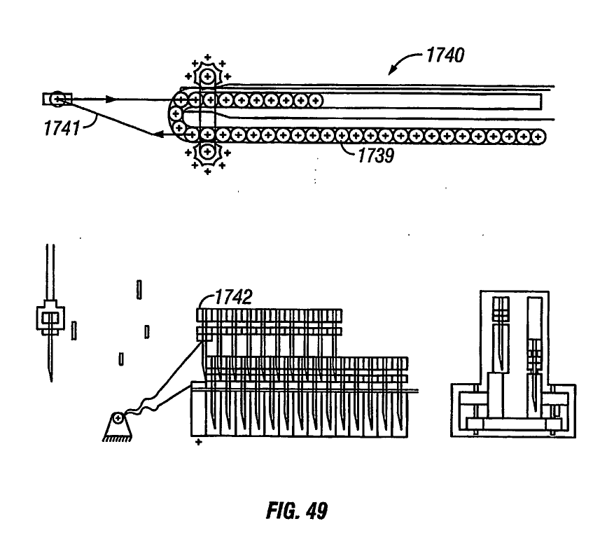

Referring to the embodiment of FIG. 49, penetrating members 1739 are stored in an oval channel 1740, tightly packed so that used penetrating members urge fresh penetrating members into the feed mechanism. The feed mechanism 1741 engages the round caps 1742 on the ends of the penetrating members. Cogs on both the new and used penetrating member tracks advance the penetrating member caps by rotating, as a gear would engage a rack. The penetrating member loading mechanism lifts a fresh penetrating member out of its cap and transports it to the launch mechanism for actuation. The used penetrating member may then placed above an empty cap in the used penetrating member channel which stores used penetrating members above used caps without engaging the penetrating members with the caps. The loading mechanism then lowers to pick up the next new penetrating member, completing the cycle. This provides a non-belt but channel based penetrating member transport device.

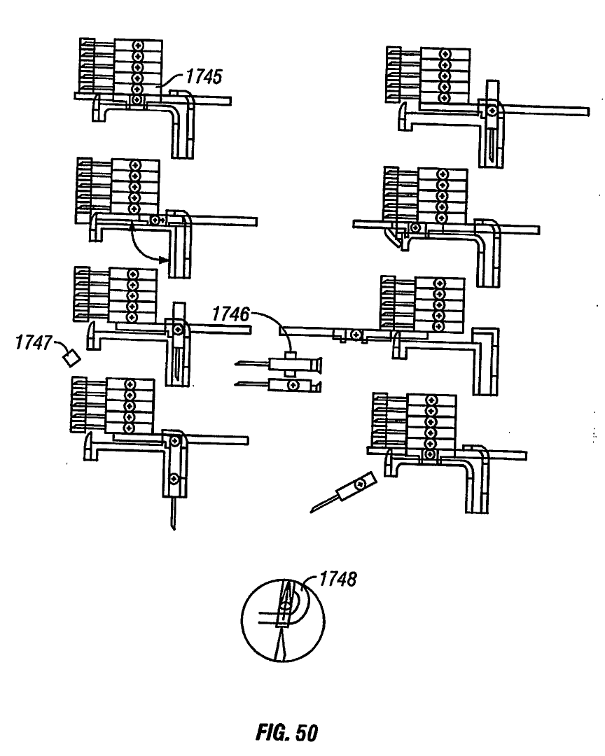

Referring to the embodiment of FIG. 50, penetrating members 1745 are stored in a stack and drawn out by a mechanism that grips lugs 1746 on the side of the penetrating member overmold housing. A cap 1747 is removed from the penetrating member prior to actuation. At the end of the extraction stroke, the penetrating member is rotated about 90 degrees or some angle sufficient to align with the actuator, and the mechanism moves clear of the actuator. The used penetrating member may be picked up by the mechanism, rotated 90 degrees or some other angle sufficient to get the penetrating member back to its original orientation, and pushed past the clean penetrating members into a discard stack or bin. Also shown is a study of a concept where two lugs are used on either side of the penetrating member body. The lugs 1746 follow a cam-type groove 1748 that produces the rotation. Any other mechanism useful for rotating a penetrating member 1745 may also be used in place of the lugs 1746 and groove 1748 combination.

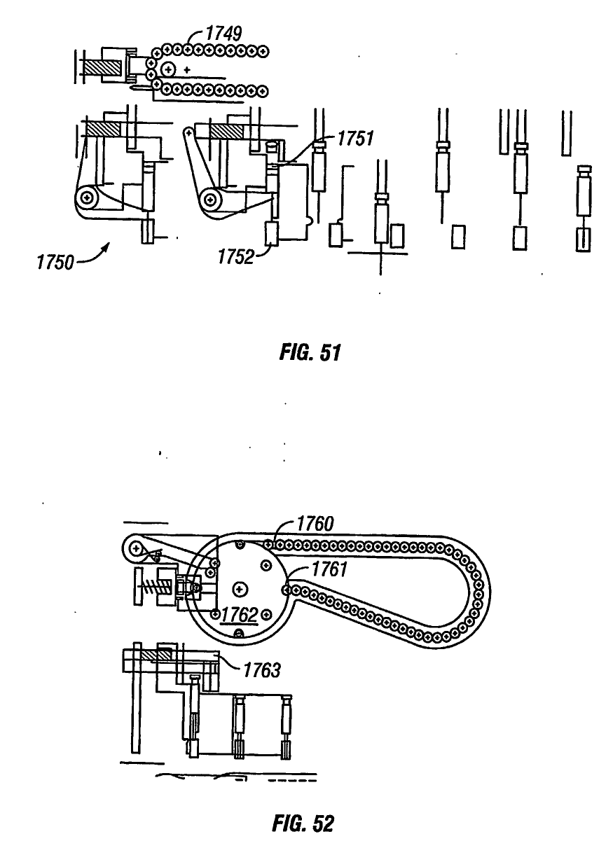

Referring to the embodiment of FIG. 51, this actuator is similar to that of FIG. 23 in that penetrating members are stored in a channel and are lifted out of their protective caps by a transfer mechanism that carries the penetrating member to the actuation mechanism and releases it. Used penetrating members are transported back to the penetrating member storage channel and released. A fresh penetrating member is then rotated into position to complete the cycle. In FIG. 51, the transfer mechanism 1750 is shown in more detail, and the penetrating member advance is by use of a single cogwheel 1749. FIG. 51 shows the removal of a penetrating member 1751 from the protective cap 1752.

Referring to the embodiment of FIG. 52, penetrating members are stored in a channel 1760 that fits tightly enough to urge new penetrating members 1761 into an indexing disc 1762. The indexing disc 1762 carries new penetrating members to a position where a transfer mechanism 1763 can pick the penetrating member from the disc and place it into the launch mechanism then move clear. A used penetrating member is picked up by the transfer mechanism and placed back in the indexing disc 1762. The indexing disc 1762 inserts the used penetrating member into the storage channel displacing a new penetrating member into the transfer disc and completing the cycle.

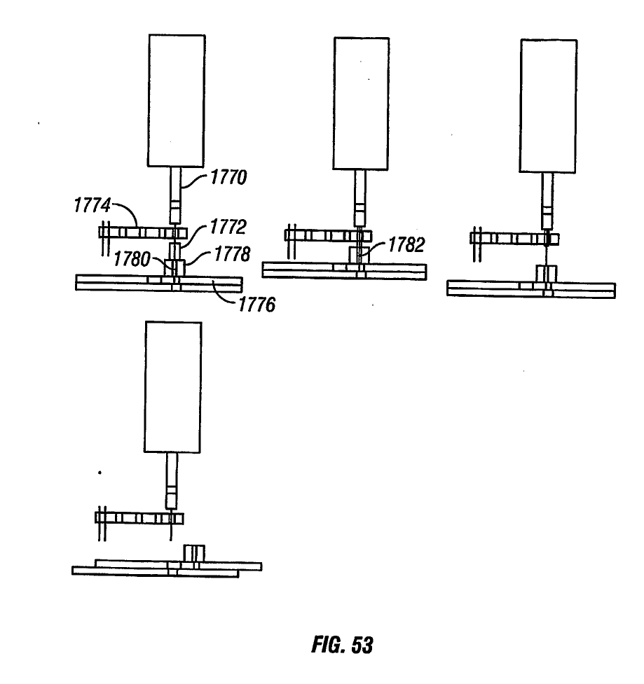

Referring to the embodiment of FIG. 53, penetrating members 1770 with molded-on caps 1772 are stored on the periphery of a disc. The disc 1774 is rotated to place a fresh penetrating member into the launching mechanism. A sub plate 1776 with a press surface 1778 for the cap, and a clearance hole 1780 for the penetrating member is moved under the cap and forces the cap up the penetrating member to expose a sterile penetrating member and tip 1782. The sub plate moves out of the way, and the penetrating member may be actuated. The carrier disc indexes a new penetrating member into position completing the cycle.

In other embodiments, this transfer mechanism is very similar to that shown in FIG. 50 except that the penetrating member body is rotated by means of gear teeth molded into the lugs on the side of the penetrating member body. The penetrating member lugs are moved over a fixed rack causing a 90-degree rotation, or moving racks are built into the grip and transfer mechanism and are actuated independently to produce the rotation. In another embodiment, this transfer mechanism is very similar to that shown in FIG. 49 except that the entire penetrating member storage cartridge moves under the penetrating member launch mechanism, raises to insert a used penetrating member into its cap, indexes a new penetrating member onto the drive, drops down to remove the penetrating member from its cap, then moves away leaving the penetrating member freely engaged in the launching mechanism.

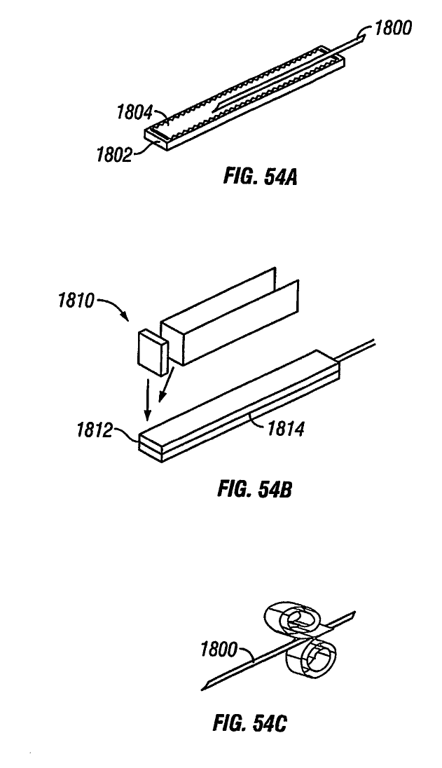

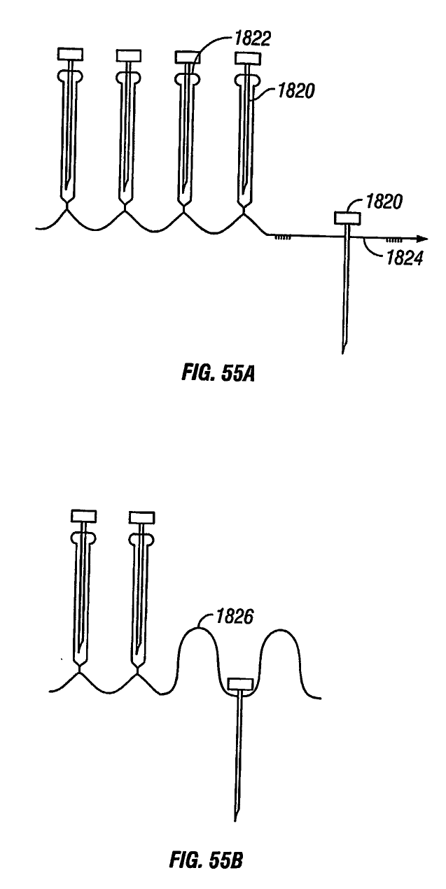

For any of the above embodiments of the present invention, penetrating members enclosed in the following enclosure may also be used or adapted for use with feed or transport mechanisms disclosed in the present application. As seen in FIG. 54A, penetrating member 1800 may be covered with strips 1802 of material such as but not limited to metal, plastic, elastomer, or other enclosure material that are preformed into a coiled state. The top and bottom protective strips are forced flat and may be laminated, adhered, or otherwise attached to surround either side of the penetrating member 1800 to provide a sterile environment for at least those portions of the penetrating member requiring such care. A light adhesive 1804 may be used to seal the penetrating member 1800 therein. In some embodiments, a heavier, more durable, adhesive could be used at the point where the shaft of the penetrating member passes out of the lamination. A heavier, friable, adhesive may be used to bond the ends of the protective strips close to the penetrating member tip. Individual penetrating members may be prepared in large quantities on continuous strips of tape, then cut into individual assemblies or used in tape form after partial perforation.

In use, the protected penetrating member 1800 is advanced into an actuating mechanism in preparation for launch. As seen in FIG. 54B, a portion of the transfer mechanism or a penetrating member release device 1810 crushes the friable adhesive at the tip 1812 of the protective strip assembly. The penetrating member release device 1810 may assume a variety of different shapes, such a blade configuration, a U-shaped blade for impacting a greater area of the penetrating member enclosure, or other suitable shapes for releasing friable bond used on the enclosure. For illustration purposes, FIG. 28B shows two types of penetrating member release devices 1810. The fractured adhesive loses its bond strength and allows the cover strips to begin to curl into their coiled state as seen in FIG. 54C. In one embodiment, the weaker adhesive on the sides 1814 of the cover strips fails progressively as the covers regain their coiled shape. In other embodiments, strong adhesive may also be used on the sides and thus a shaped blade such as but not limited to the U-shaped blade of FIG. 54B may be used. The process ends when the heavier adhesive is reached, and the strips are fully relaxed. The coiled strips are retained on the penetrating member shaft for disposal. The adhesive to be used is sufficient so that a sterile bond can be maintained, and the side adhesive release completely after sterilization. Suitable strong adhesives include, but are not limited to, PMMA, cyanoacyrlate, latex, or other materials useful for such adhesion. Additionally, in some embodiments, only one layer has a shape memory or other feature that causes the layer to coil and reveal the penetrating member. The coil in some embodiments may occur substantially orthogonal to the longitudinal axis or via a tear down the mid-line or centerline of the enclosure instead of from the edges.

For any of the above embodiments of the present invention, penetrating members enclosed in the following enclosure may also be used. Individual penetrating members 1820 are sealed in packets 1822 in a single continuous strip or tape 1824 of flexible material such as but not limited to paper, plastic, polymeric material, Tyvek, rubber, elastomer, or other suitable material. The tape may also have a silicone or other material coating. The tape may further includes a lead in or lead out to facilitate loading. The packets are formed by piercing the penetrating member 1820 through a ribbon of tape 1824 at the centerline or some other location. Either the dull, or the sharpened end of the penetrating member could pierce the tape, or the tape could be pre-punched with a hole to allow the penetrating member to pass through. The penetrating member 1820 may then be bonded to the tape to establish its position and to seal between the tape and the penetrating member. The tape is then folded down along both sides of the penetrating member and bonded to itself forming a closed pouch 1822 containing the penetrating member. The process is then repeated, resulting in a series of penetrating members 1820 in packets 1822 connected by a “U” fold of tape between each packet.

In use as seen in FIG. 54A, the loading mechanism may grasp the tape 1824 on either side of a penetrating member 1820 and pull them apart, peeling open the packet 1822. The fresh, sterile, penetrating member 1820 is now suspended in the middle of a section of tape. The loading mechanism transports the penetrating member into the launcher drive chuck and relaxes the tape to allow a flexible loop 1826 on each side of the penetrating member as seen in FIG. 54B. The penetrating member 1820 is now free to move through the actuation cycle under the control of the driver (not shown). If the tape is bonded slightly above the point of maximum penetrating member penetration, it will act as a shield to protect the actuator mechanism from contamination with blood. Any blood that may contact the tape will be cleared away with the next index cycle.

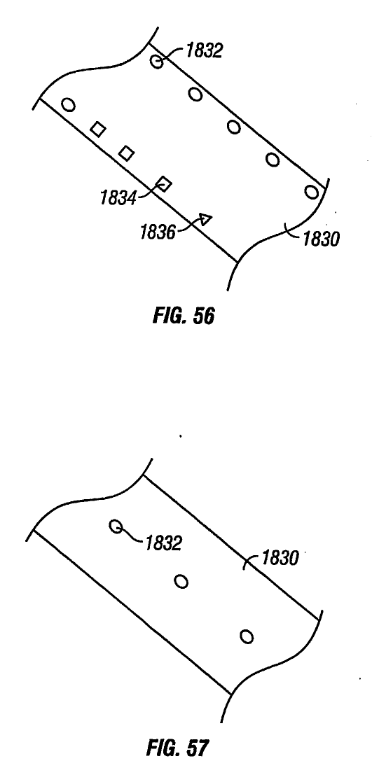

Referring now to FIGS. 56 and 57, embodiments of tapes used with the present invention may include a plurality of holes or openings to assist in guiding the tape. As seen in FIG. 56, the tape 1830 may have circular openings 1832, square openings 1834, triangular openings 1836, or other shape suitable for guiding and/or tractoring the tape. The openings may be in two or more rows or may be in a single row as shown in FIG. 40. These openings may be formed in the tape 1830 prior to assembly with penetrating members or may be formed in the tape after having penetrating members positioned at least partially therein. The holes may be used for alignment of the tape with the penetrating member actuation or transport mechanisms. The holes may be used to advance the penetrating members in a tractoring motion or to maintain tension in the penetrating member tape 1830. These examples are purely exemplary and some, none, all, or any combination of the above elements may be included on an embodiment of the tape.

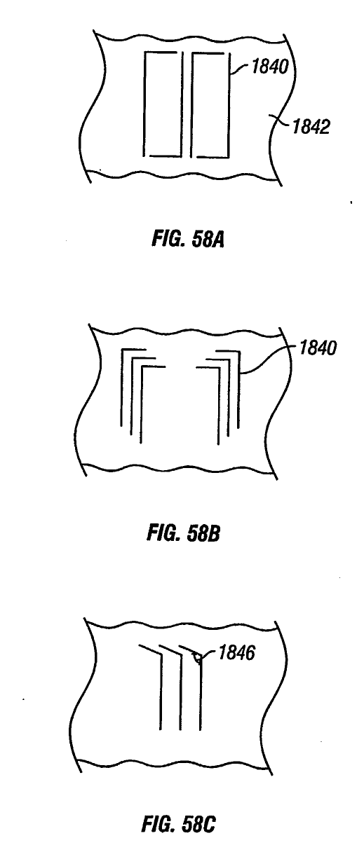

Referring now to FIGS. 58A-58C, penetrating members 1840 may be arranged in various configurations on the tape 1842 to maximize density on the tape. These figures show the penetrating members as bent penetrating members, but it should be understood that other shaped penetrating members may also benefit from these configurations. FIG. 58A shows bent penetrating members 1840 in alternating orientations. FIG. 58B shows a plurality of bent penetrating members 1840 positioned so that their contours overlap, with their bent portions facing forward, backward, or some combination of the two. FIG. 58C shows a configuration using a less pronounce bend with an angle 1846 greater than 90 degrees. The packets, blisters, or enclosures on the tape that house the penetrating members may also be in any shape useful for containing at least a portion of the penetrating member in a sterile environment. In some embodiments, the enclosure houses the entire penetrating member. In other embodiments, the enclosure may be shaped to have indentations or recessed portions conforming with cogs or protrusions on driving rollers or the like.

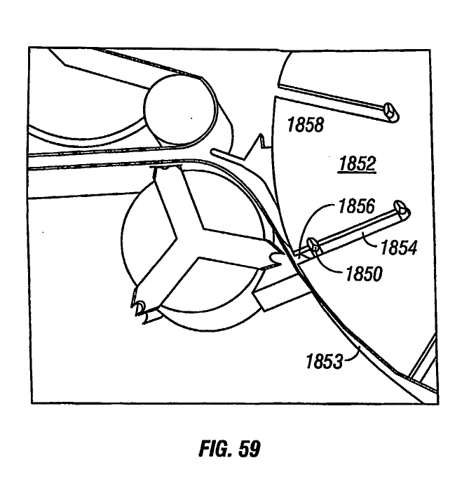

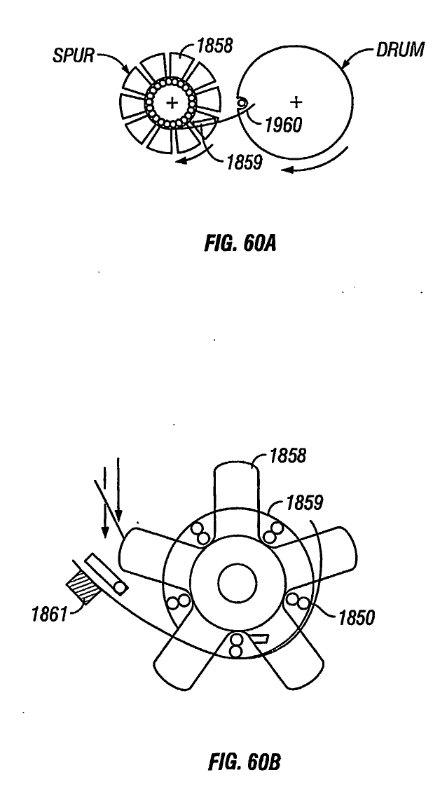

Referring to the embodiments of FIGS. 59-60, still further embodiments of penetrating member storage drums will be described. As seen in FIG. 59, the used penetrating members 1850 may be stored in a rotating drum made up of two spur shaped disks 1852 on a common shaft. Each disk has a number of radial slots that may be slightly wider than the diameter of the penetrating members. A transfer mechanism, such as but not limited to the motion of the protective tape 1853, moves a used penetrating member from the launcher mechanism into one of the radial slots 1854. The spur disks 1852 are driven by the advance mechanism. Once in the radial slot, the penetrating member is moved toward the inside of the spur by guide surfaces 1856 that are part of the housing, and by one or several spiral-shaped springs (metal or plastic) that are part of, or mounted to, the housing.

In FIG. 59, the penetrating members 1850 have formed legs or bent portions at the drive end. FIG. 59 includes the idea of using the carrier tape to transfer the penetrating members into the storage wheel and keep them there by wrapping around the outside. The guide surfaces 1856 and springs (see FIGS. 60A-60B) align the formed legs in the trailing direction, and in a circumferential orientation, to prevent them from jamming with the next layer of penetrating members as it is moved into place. The collection spurs 1858 are synchronized with the drive mechanism so that they rotate in one direction only and load used penetrating members sequentially into the storage slots 1854 until all of the penetrating members have been used and are stored in the collection spurs. The used penetrating members are retained in their slots 1854 by the action of the spiral-shaped springs and by the used protective tape that wraps around the outside of the collection spurs 1858. The collection spurs 1858 may be contained inside a plastic housing that is sealed to prevent access to the penetrating members. This sealed container, or cassette, is removed and disposed of as contaminated waste after all of the penetrating members have been used.

Referring now to FIGS. 60A and 60B, the springs used to hold the penetrating members in the slots 1854 are now described. The springs 1859 may be used to guide the used penetrating members into the slots 1854 and then urge them towards the center of the disk 1852. Multiple springs (two are shown in FIG. 60A) may be used to retain the penetrating members at the center of the spurs without excessive friction. In one embodiment, in place of the guide surfaces and protective tape transfer, the spiral-shaped springs could have an extended portion 1860 extending into a groove in the launching mechanism to scoop up the used penetrating member and direct it into the collection spurs similar to that shown in FIG. 60A. Additionally, the arms of the spurs may be constructed with a hook shape that can scoop used penetrating members directly from the launching mechanism and into the spiral-shaped springs for retention. The spring 1859 may also be attached or slidably attached so that the portion 1861 will be positioned to engage used penetrating members as they come off the actuation device and portion 1861 will guide them into the slots 1854. In other embodiments, the spurs may engage the penetrating members first, and although the spring 1859 will remain in a position sufficient to engage the used penetrating members at some point so that the penetrating members are urged towards the center of the slots 1854. It should be understood that any of the feed mechanism described herein may be adapted for use with any of the penetrating member drivers disclosed herein including but not limited to those in FIGS. 1-3.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. For example, with any of the embodiments discussed above, another cutting blade or release device may be used to release the penetrating member prior to loading. Such a blade may, though not necessarily, cut the enclosure in a manner parallel to the tape surface. With any of the above embodiments, the location of the penetrating member loading or release devices may be changed (such as but not limited to being mounted on or coordinated with an upper roller, instead of a lower roller). The penetrating member enclosure may be of the vacuum sealed variety, where there is no space, bubble, or defined area between the penetrating member and the sealing layer. Expected variations or differences in the results are contemplated in accordance with the objects and practices of the present invention. Variations in skin thickness including the stratum corneum and hydration of the epidermis can yield different results between different users with existing tissue penetration devices, such as but not limited to lancing devices wherein the tissue penetrating element of the tissue penetration device is a penetrating member. The rollers and tapes may be aligned in a variety of positions to transport the tape left to right, right to left, up to down, down to up, front to back, or other orientation useful for bringing penetrating members from a storage area to a launch area. The penetrating members may be designed to travel on a curved path into the tissue. The penetrating members may be curved penetrating members. The penetrating members maybe coated or uncoated. The penetrating members may have certain frictional properties. The tapes may be held in a circular configuration or in a plurality of rigid concentric circular arrays. The penetrating member loading mechanisms may be run in a continuous pick off mode or in a reciprocating pick off mode where appropriate. The drum may have other configurations instead of round, such as but not limited to but not limited, to square, rectangular, polygonal, triangular, or other configuration. The drive chuck may allow for off-center actuation, multiple chucks may be used, or the like. The rollers may have a concave, convex, or other deliberately varying profile so as to improve tension control or other operational feature of the system. The penetrating member may bend a little bit (catching post) so that the penetrating member is (in one embodiment) always into the chuck. Unwanted resistance in transporting the tape may involve a choice of materials, surface finishes, tape material, tape material, tape thickness, and total peel strength to pull apart. To make sure there is no vertical wander of the tape, the tape may be pre aligned when it goes into the system. To stop it from riding up or down, the transfer drum may be contoured. The transfer drum may be an arc, not a complete circle. The device may be gravity independent. A floor and ceiling of casing may be provided to prevent penetrating member from falling out of its retained position. The tissue penetrating system may have a window for viewing status of the cartridge or the penetrating members.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

This application claims the benefit of priority from commonly assigned, copending U.S. Application Ser. Nos. 60/437,359, 60/437,205, 60/478,661, fully incorporated herein by reference for all purposes. The publications discussed or cited herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed. All publications mentioned herein are incorporated herein by reference to disclose and describe the structures and/or methods in connection with which the publications are cited.

Expected variations or differences in the results are contemplated in accordance with the objects and practices of the present invention. It is intended, therefore, that the invention be defined by the scope of the claims which follow and that such claims be interpreted as broadly as is reasonable.

1. A body fluid sampling system comprising:
a penetrating member driver;
a plurality of penetrating members sufficient for penetrating tissue;
a tape coupling together at least two of said penetrating members;
a penetrating member release device removing the penetrating member from a sterile environment prior to use and moving said penetrating member into position to be operatively coupled to said penetrating member driver.

2. The system of claim 1 wherein said release device comprises a rotating member having a portion of sufficient sharpness to at least partially penetrate said tape and a portion shaped to engage said penetrating member, said rotating member movable to urge said penetrating member to engage a coupler on the penetrating member driver.

3. The system of claim 1 wherein said release device comprises a rotating member having a portion of sufficient sharpness to penetrate a penetrating member enclosure.

4. The system of claim 1 wherein said release device comprises a movable member sufficient to pierce a penetrating member enclosure, engage the penetrating member, and moving said penetrating member to engage a coupler on the penetrating member driver.

5. The system of claim 1 further comprising a penetrating member unloading device to remove said penetrating member from the penetrating member driver.

6. The system of claim 1 wherein said penetrating members comprise a unitary body.

7. The system of claim 1 wherein said penetrating members are without molded attachments.

8. (canceled)

9. A tissue penetrating system for use with a plurality of penetrating members, the tissue penetrating system comprising:
a penetrating member driver;
a penetrating member release device removing one of the penetrating members from a sterile environment prior to use;
a penetrating member loading device receiving penetrating members from the release device, said loading device moving said penetrating member to be operatively coupled to said penetrating member driver.

10. The system of claim 9 wherein said loading device comprises a transfer drum having an area shaped to receive one of said penetrating members.

11. The system of claim 9 wherein said loading device comprises a transfer drum having an opening for receiving one of said penetrating members.

12. The system of claim 9 further comprising penetrating member unloading device for moving said penetrating member from the coupler to a storage canister.

13. A tissue penetrating system for use with a plurality of penetrating members, the tissue penetrating system comprising:
a penetrating member driver;
a penetrating member transport device;
a penetrating member loading device receiving penetrating members from the transport device, said loading device moving said penetrating member to be operatively coupled to said penetrating member driver;
wherein said penetrating member transport device is configured to receive said penetrating members in a sealed condition and to deliver said penetrating members in an unsealed condition to the penetrating member loading device.

14. The system of claim 13 wherein said penetrating member transport device uses a plurality of rollers positioned to advance the penetrating members and to remove each one from a sealed condition prior to reaching the penetrating member loading device.

15. The system of claim 13 wherein said penetrating member transport wherein said loading device includes a surface configured for slidably engaging said penetrating member from the transport device.

16. The system of claim 13 wherein said penetrating member transport wherein said loading device includes a surface configured for slidably engaging said penetrating member from the transport device, said surface being a hole and an L-shaped penetrating member or a penetrating member with orthogonal orientation.

17. A tissue penetrating system for use with a penetrating member driver and a plurality of penetrating members, said system comprising:
a tape for holding said penetrating members;
a loading device for moving said penetrating member into position to be coupled to the driver;
a peel device for removing an active one of said penetrating members from said tape;
a tape tension device coupled to the peel device for maintaining said penetrating member and synchronizing said penetrating members with said loading device.

18. The system of claim 17 wherein said penetrating members are at a fixed spacing.

19. The system of claim 17 wherein said penetrating member transport device uses a plurality of rollers positioned to advance the penetrating members and to remove each one from a sealed condition prior to reaching the penetrating member loading device.

20. A tissue penetrating system for use with a penetrating member driver and a plurality of penetrating members, said system comprising:
a penetrating member transport device;
a penetrating member loading device receiving penetrating members from the transport device, said loading device moving said penetrating member to be operatively coupled to said penetrating member driver;
a tape peeling assembly peeling said tape apart into a first portion and a second portion, said first portion peeled apart at a selected peel angle relative to the second portion.

21. The system of claim 20 wherein said tape peeling assembly prevents said tape from jamming by maintaining a consistent tension when the tape is being advanced and peeled apart.

22. The system of claim 20 wherein said tape peeling assembly provides a sufficient tension when the tape is being advanced so that the peel point does not change in a manner that the penetrating members no longer align with receiving areas on the loading device.

23. The system of claim 20 wherein said tape peeling assembly maintains a consistent spacing between penetrating members as the members are coupled to the loading device.

24. The system of claim 20 wherein said tape peeling assembly maintains a consistent spacing between a first penetrating member to be removed from the tape and coupled to the loading device, and a second penetrating member to be coupled to the loading device after the first penetrating member is loaded.

25. The system of claim 20 wherein said tape peeling assembly includes at least one piercing blade for piercing said tape and engaging a first penetrating member to be coupled to the loading device.

26. The system of claim 20 wherein said tape peeling assembly rotates sufficiently to compensate for a slight path difference between the tape and the penetrating member about said loading device, said assembly being tensioned up during advancement of the tape to remove slack in the tape that may alter the peel point.

27. The system of claim 20 wherein said tape includes a plurality of tractor holes.

28. The system of claim 20 wherein said tape peeling assembly is coupled to a differential that tightens the tension to a predefined level and slips if the user winds the differential to tension beyond the predefined level.

29. The system of claim 20 wherein said tape are adhered together in manner such that the tape is peeled apart in a consistent manner.

30. The system of claim 20 wherein said blade on the loading device is above the external stroke of the penetrating member, so said penetrating member is kept clean.

31-37. (canceled)

38. A tissue penetrating system for use with at least one penetrating member, the tissue penetrating system comprising:
a penetrating member driver;
a penetrating member release device removing the penetrating member from a sterile environment prior to use and moving said penetrating member into position to be operatively coupled to said penetrating member driver.

39. The system of claim 38 wherein said penetrating members are bare penetrating members or without attachments.

40. The system of claim 38 wherein said release device comprises a rotating member having an outer portion of sufficient sharpness to penetrate penetrating member enclosure and a portion shaped to engage said penetrating member, said rotating member movable to urge said penetrating member to engage a coupler on the penetrating member driver.

41. The system of claim 38 wherein said release device comprises a movable member sufficient to pierce a penetrating member enclosure, engage the penetrating member, and moving said penetrating member to engage a coupler on the penetrating member driver.

42. The system of claim 38 further comprising a penetrating member unloading device to remove said penetrating member from the penetrating member driver.

39-61. (canceled)
